(19) United States
(12) Patent Application Publication
(10) Pub. No.: US 2013/0078637 A1
(43) Pub. Date: Mar. 28, 2013

(54) ANTIPSYCHOTIC-INDUCED PARKINSONISM GENOTYPES AND METHODS OF USING SAME

(71) Applicants: Bernard Lerer, Alon Shvut (IL); Lior Greenbaum, Yehud (IL); Anna Alkelai, Bet Shemesh (IL)

(72) Inventors: Bernard Lerer, Alon Shvut (IL); Lior Greenbaum, Yehud (IL); Anna Alkelai, Bet Shemesh (IL)

(73) Assignee: HADASIT MEDICAL RESEARCH SERVICES AND DEVELOPMENT LTD., Jerusalem (IL)

(21) Appl. No.: 13/700,103

(22) PCT Filed: May 26, 2011
(86) PCT No.: PCT/IL11/00411

§ 371 Date: Nov. 26, 2012

Related U.S. Application Data
(60) Provisional application No. 61/348,759, filed on May 27, 2010.

(51) Int. Cl. C12Q 1/68

(52) U.S. Cl. CPC C12Q 1/68
USPC 435/6.11

(57) ABSTRACT

The present invention relates to genotypes associated with resistance to antipsychotic-induced parkinsonism and other extrapyramidal symptoms induced by antipsychotics, and use of said genotypes for assessment of patient populations. The methods and kits of the invention are based on identifying in a sample obtained from a subject, specific SNPs in the ZFPM2 and RGS2 genes.

### FIELD OF THE INVENTION

The present invention relates to genotypes associated with resistance to antipsychotic-induced parkinsonism and other extrapyramidal symptoms induced by antipsychotics, and use of said genotypes for assessment of patient populations. The methods and kits of the invention are based on identifying in a sample obtained from a subject, specific SNPs in the ZFPM2 and RGS2 genes.

### BACKGROUND OF THE INVENTION

The use of antipsychotic, neuroleptic drugs is associated with the development of extrapyramidal symptoms (EPS) which may be acute and reversible (such as, dystonia, parkinsonism, and akathisia) or long lasting and chronic (e.g. tardive dyskinesia and also dystonia). EPS are a major problem specifically in schizophrenia treatment due to their negative effect on adherence to treatment, patient distress, social stigma, and reduced quality of life.

Although atypical antipsychotic agents (also known as second generation antipsychotics) have a significantly decreased propensity to cause extrapyramidal side effects, they do not provide total relief to every psychotic patient. Improvement in the clinical efficacy of atypical antipsychotics is achieved, in certain patients, by combining these drugs with other antipsychotics, including typical antipsychotics, thereby exposing patients to onset or worsening of EPS.

A method for the determination of the severity of drug induced EPS, irrespective of the subject, is disclosed in US patent application, publication No. 2006/0252103. The method comprises determining the pattern of differential internalization of a receptor, in cell lines expressing the receptor, caused due to binding of said drugs with said receptor, and correlating said internalization pattern with the severity of EPS.

Methods for assessing a tendency or resistance of a subject to develop EPS following treatment with antipsychotic drugs, based on the presence of several SNPs and specific haplotypes in the nucleotide sequence of RGS2, a regulator of G-protein signaling, are disclosed in International application, PCT publication No. WO 2007/144874, by the inventors of the present invention. WO 2007/144874 is expressly incorporated herein by reference in its entirety.

Antipsychotic-induced parkinsonism (AIP) is the most common manifestation of EPS. Interindividual heterogeneity in AIP development and severity is associated with risk factors such as antipsychotic drug type, old age, and female gender. However, there is evidence for genetic predisposition to develop AIP, but the variants that confer susceptibility or protection are mostly unknown.

Methods for determining the predisposition of an individual to two or more phenotypes related to pediatrics or reproduction, suitability for military service and longevity, wherein one of the phenotypes is AIP, are disclosed in US patent applications, publication Nos. US 2009/0307181; US 2009/0307180 and US 2009/0307179, respectively. The methods associate the predisposition with the presence of a specific set of genetic variants in genetic material obtained from said individual. These methods do not attempt to evaluate predisposition of an individual to drug induced EPS, specifically, to AIP.

Several phenotypes related to protection from or susceptibility to AIP were recently published by the inventors of the present invention (Alkelai et al., Psychopharmacology, Aug. 13, 2009, 206:491-49). These phenotypes include SNPs in genes that are not known, to date, to be associated with AIP, idiopathic Parkinson's disease (PD) or schizophrenia. Moreover, these findings are the first case-control, pharmacogenomic genome-wide association study (GWAS) for AIP severity. This publication is incorporated herein, in its entirety, by reference.

There is an unmet need for determining AIP susceptibility prior to treatment with antipsychotic neuroleptic drugs, which will enable optimization of antipsychotic treatment regimen.

### SUMMARY OF THE INVENTION

The present invention provides methods and kits for assessing phenotypes that are resistant to development of extrapyramidal symptoms (EPS), including, antipsychotic-induced Parkinsonism (AIP) or for EPS, such as, AIP, to worsen, upon treatment with antipsychotic drugs.

The methods of the invention are based in part on the unexpected discovery of a specific SNP, namely, rs12678719 in ZFPM2 gene, which highly associates with resistance to EPS and Parkinsonism induced by typical antipsychotics. The SNP rs12678719 on ZFPM2 was previously listed among 14 other SNPs, to be related to AIP susceptibility or resistance (Alkelai, ibid). However, a comprehensive validation analyses led to the discovery that the only one SNP with strong association to AIP is rs12678719 on ZFPM2 (Greenbaum et al, to be Submitted). The present invention provides additional predictive means, namely, rs4606 in RGS2 gene, which in combination with rs12678719 on ZFPM2 bolsters the assessment achieved by the methods and kits of the invention. As detailed below, the prediction made by the methods of the invention apply not only to subjects having the c allele of rs12678719 in the ZFPM2 gene, but also to subject that do not have that allele but rather have the g allele of rs4606 in the RGS2 gene. Accordingly, the present invention provides for the first time a strong predictive platform, to help the physicians deciding whether to prescribe typical (conventional) antipsychotics to a subject in need.

The inventors further establish herein that association of specific SNPs is unpredictable, even if the genes encompassing the SNPs are known to be associated with AIP, schizophrenia or idiopathic Parkinson's disease. As exemplified below, the inventors of the present invention have found that genes which based on the art are expected to be associated with AIP, do not show such association.

The aforementioned surprising discoveries are a result of using the advantageous genome-wide pharmacogenomic approach (also termed hereinafter, “GWAS”), which allows unbiased, “hypothesis-free” detection of DNA variants associated with the phenotype of interest.

It is to be understood that according to the principles of the present invention the terms “extrapyramidal symptoms” or “EPS” refer to extrapyramidal symptoms induced by typical (conventional) antipsychotics. These terms are interchangeable with any extrapyramidal symptoms induced by typical (conventional) antipsychotics, including, but not limited to, antipsychotic-induced parkinsonism (AIP), antipsychotic-induced dystonia and antipsychotic-induced akathisia.

According to one aspect, the present invention provides a method for assessing resistance of a subject to develop antipsychotic-induced parkinsonism, comprising:
(a) obtaining a sample comprising genetic material from the subject;
(b) determining in said genetic material the presence of the nucleotide sequence of the ZFPM2 gene or a fragment thereof; and
(c) identifying in said nucleotide sequence the polymorphic site rs12678719,

wherein the presence of cytosine at the polymorphic site rs12678719 is indicative of resistance to emergence or aggravation of antipsychotic-induced parkinsonism.

As used herein, the term “develop” encompasses the emergence and/or aggravation of the antipsychotic-induced parkinsonism (AIP) following treatment with an antipsychotic drug, such that these terms are used herein interchangeably.

According to one embodiment, the method further comprises determining in said genetic material the presence of the nucleotide sequence of the RGS2 gene or a fragment thereof; and identifying in said nucleotide sequence the polymorphic site rs4606, wherein the presence of guanine at rs4606 is indicative of resistance to emergence or aggravation of antipsychotic-induced parkinsonism.

According to another embodiment, the method comprises determining in said genetic material the presence of a first nucleotide sequence comprising the ZFPM2 gene or a fragment thereof and a second nucleotide sequence comprising the RGS2 gene or a fragment thereof; and identifying in said first and second nucleotide sequences the polymorphic sites, rs12678719 and rs4606, respectively, wherein the presence of cytosine at rs12678719 or guanine at rs4606 is indicative of resistance to emergence or aggravation of antipsychotic-induced parkinsonism.

According to yet another embodiment, the presence of cytosine at rs12678719 and guanine at rs4606 is indicative of resistance to emergence or aggravation of antipsychotic-induced parkinsonism.

According to another aspect, the present invention provides a method for assessing resistance of a subject to develop extrapyramidal symptoms upon treatment with one or more antipsychotic drugs, comprising:
(a) obtaining a sample comprising genetic material from the subject;
(b) identifying in said genetic material the polymorphic sites rs12678719 in the ZFPM2 gene and rs4606 in the RGS2 gene; and
(c) analyzing the results,

wherein the presence of at least one of cytosine at rs12678719 and guanine at rs4606 is indicative of resistance to emergence or aggravation, of extrapyramidal symptoms induced by treatment with the one or more antipsychotic drugs.

According to some embodiments, determining, the presences of the polymorphic sites in the genetic material obtained in the methods of the invention, comprises amplifying the genetic locus encompassing said at least one polymorphic site.

According to certain embodiments, the sample is obtained from a biological specimen selected from the group consisting of: blood, saliva, urine, sweat, buccal material, skin and hair.

Any method for determining nucleic acid sequence and for analyzing the identified nucleotides for polymorphism, known to a person skilled in the art, can be used according to the teachings of the present invention.

According to certain embodiments, identifying the at least one site of nucleotide polymorphism is attained by a technique selected from the group consisting of: terminator sequencing restriction digestion, allele-specific polymerase reaction, single-stranded conformational polymorphism analysis, genetic bit analysis, temperature gradient gel electrophoresis, ligase chain reaction and ligase/polymerase genetic bit analysis.

According to yet another embodiment, the nucleotide polymorphism is identified by employing nucleotides with a detectable characteristic selected from the group consisting of inherent mass, electric charge, electric spin, mass tag, radioactive isotope type bioluminescent molecule, chemiluminescent molecule, hapten molecule, protein molecule, light scattering/phase shifting molecule and fluorescent molecule.

According to yet another embodiment, the subject in need thereof is psychotic. According to yet another embodiment, the subject in need thereof is diagnosed with schizophrenia.

According to yet another embodiment, the method for diagnosing the resistance to emergence or aggravation of antipsychotic-induced parkinsonism is performed prier to initiation of treatment with one or more antipsychotic drug.

According to yet another embodiment, the method is performed after initiation of the treatment with one or more antipsychotic drug.

According to yet another embodiment, the antipsychotic drug is selected from the group consisting of: perphenazine, olanzapine, clozapine, quetiapine, resperidone and ziprasidone alone or in combination with one or more antipsychotic drug.

According to yet another embodiment, the method further comprises repeating steps (b) and (c). According to yet another embodiment, the method further comprises amplifying said nucleotide sequence of the gene or fragment thereof prior to step (c).

According to yet another embodiment, the method is directed to antipsychotic-induced parkinsonism comprising one or more of bradykinesia, tremor, rigidity, stooped posture, gait disturbance, salivation and seborrheic dermatitis.

According to yet another aspect, the present invention provides a kit for assessing resistance of a subject to develop API, comprising oligonucleotides for amplification of the genetic locus encompassing the polymorphic site rs12678719 in the gene ZFPM2 within a sample obtained from the subject.

According to one embodiment, the kit further comprises means for determining the presence of the c allele of rs12678719, which indicates that said subject is resistant to development of extrapyramidal symptoms induced by treatment with antipsychotics.

According to another embodiment, the kit further comprises oligonucleotides for amplification of the genetic locus encompassing the polymorphic site rs4606 in the gene RGS2 within said sample.

According to one embodiment, the kit further comprises means for determining the presence of the g allele of rs4606, which indicates that said subject is resistant to development of extrapyramidal symptoms induced by treatment with antipsychotics.

According to one embodiment, the sample is obtained from a biological specimen selected from the group consisting of: blood, saliva, urine, sweat, buccal material, skin and hair.

According to another embodiment, the subject is psychotic. According to yet another embodiment, the subject is diagnosed with schizophrenia.

Other objects, features and advantages of the present invention will become clear from the following description and drawings.

### DETAILED DESCRIPTION OF THE INVENTION

The present invention provides methods and kits for assessing phenotypes that are resistant to development of extrapyramidal symptoms (EPS), including, antipsychotic-induced parkinsonism (AIP) or for EPS, such as, AIP, to worsen, upon treatment with antipsychotic drugs. The methods and kits are based on the identification of the SNP rs12678719 (ZFPM2 gene) alone or together with the identification of the SNP rs4606 (RGS2).

### DEFINITIONS

As used herein, the term “gene” has its meaning as understood in the art. In general, a gene is taken to include gene regulatory sequences (e.g. promoters, enhancers, etc.) and/or intron sequences, in addition to coding sequences (open reading frames). It will further be appreciated that definitions of “gene” include references to nucleic acids that do not encode proteins but rather encode functional RNA molecules such as microRNAs (miRNAs), tRNAs, etc.

The term “allele” refers to an alternative version (i.e., nucleotide sequence) of a gene or DNA sequence at a specific chromosomal locus.

The term “polymorphism” as used herein refers to the occurrence of two or more alternative genomic sequences or alleles in a population. “Polymorphic” refers to the condition in which two or more variants of a specific genomic sequence can be found in a population. A “polymorphic site” is the locus at which the variation occurs. Polymorphism refers to the occurrence of two or more genetically determined alternative sequences or alleles in a population. Preferred polymorphisms have at least two alleles, each occurring at frequency of greater than 1%, and more preferably greater than 10% or 20% of a selected population. A polymorphic locus may be as small as one base pair. Polymorphic markers include restriction fragment length polymorphisms, variable number of tandem repeats (VNTRs), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wild type form. Diploid organisms may be homozygous or heterozygous for allelic forms. A biallelic polymorphism has two forms. A triallelic polymorphism has three forms.

The terms “single nucleotide polymorphisms” or “SNPs” (pronounced “snips”) are interchangeably used to describe particular DNA sequence variations that occur when a single nucleotide (A, T, C or G) in the genome sequence is altered. For example, a SNP might change the DNA sequence AAGGCTAA to ATGGCTAA. For a variation to be considered a SNP, it must occur in at least 1% of the population. SNPs, which make up about 90% of all human genetic variation, occur every 100 to 300 bases along the 3-billion-base human genome. The site is usually preceded by and followed by highly conserved sequences of the allele (e.g., sequences that vary in less than 1/100 or 1/1000 members of the populations). A single nucleotide polymorphism usually arises due to substitution, of one nucleotide for another at the polymorphic site. A transition is the replacement of one purine by another purine or one pyrimidine by another pyrimidine. A transversion is the replacement of a purine by a pyrimidine or vice versa. Single nucleotide polymorphism can also arise from a deletion of a nucleotide or an insertion of a nucleotide relative to a reference allele. It should be noted that a single nucleotide change could result in the destruction or creation of a restriction site. Therefore it is possible that a single nucleotide polymorphism might also present itself as a restriction fragment length polymorphism.

SNPs can occur in both coding (gene) and non-coding regions of the genome, including regulatory regions of genes. Many SNPs have no effect on cell function, but can predispose subjects to disease or influence their response to a drug.

The value of SNPs for finding propensity to diseases is disclosed in Pennisi et al. (Science, 1998, 281:5384) and Hegele et al. (Artherioscler. Thromb Vasc. Biol. 2002, 22:1058-1061) among others.

The term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences).

The terms “haplotype” and “SNP-based haplotype” are interchangeably used herein to describe a combination of polymorphisms (SNPs) occurring within a locus on a single chromosome (of either maternal or paternal origin). The “locus” includes the entire coding sequence. A haplotype may be used for detecting complex traits as it contains more than a single SNP. Each haplotype is a set of alleles within families and consideration of multiple closely-linked marker loci can provide a larger number of alleles than provided by usually bi-allelic single SNPs and may demonstrate association with a phenotype more effectively than the component single SNPs. A method for haplotyping is disclosed, for example, in U.S. Pat. No. 6,844,154.

The terms “trait” and “phenotype” are used interchangeably herein and refer to any visible, detectable or otherwise measurable property of an organism such as resistance to or the susceptibility to develop a disease or a disorder, specifically the susceptibility or resistance to development or aggravation of EPS during treatment with antipsychotic drugs, more specifically, resistance to or the susceptibility to develop AIP during treatment with antipsychotic drugs.

The term “haplotype tagging SNPs” also termed hereinafter htSNPs, is used to describe markers being a subset of the markers composing the group of linkage disequilibrium and haplotype diversity within a genomic region. In fact, htSNPs markers capture most of the haplotypes in a region of linkage disequilibrium. Thus, determination of htSNPs enables to retain much of the information of haplotypes by retaining only a reduced subset of markers, thereby saving on resources.

As used interchangeably herein, the term “oligonucleotides”, and “polynucleotides” include RNA, DNA, or RNA/DNA hybrid sequences of more than one nucleotide in either single chain or duplex form. The term “nucleotide” as used herein as an adjective to describe molecules comprising RNA, DNA, or RNA/DNA hybrid sequences of any length in single-stranded or duplex form. The term “nucleotide” is also used herein as a noun to refer to individual nucleotides or varieties of nucleotides, meaning a molecule, or individual unit in a larger nucleic acid molecule, comprising a purine or pyrimidine, a ribose or deoxyribose sugar moiety, and a phosphate group, or phosphodiester linkage in the case of nucleotides within ail oligonucleotide or polynucleotide. The term “nucleotide” is also used herein to encompass “modified nucleotide” which comprise at least one modification, including, for example, analogous linking groups, purine, pyrimidines, and sugars. However, the polynucleotides of the invention are preferably comprised of greater than 50% conventional deoxyribose nucleotides, and most preferably greater than 90% conventional deoxyribose nucleotides The polynucleotide sequences of the invention may be prepared by any known method, including synthetic, recombinant, ex vivo generation, or a combination thereof, as well as utilizing any purification methods known in the art.

The term “linkage disequilibrium”, or LD, is the non-random association of alleles at two or more loci. It is not the same as linkage, which describes the association of two or more loci on a chromosome with random recombination between them. LD describes a situation in which some combinations of alleles or genetic markers occur more or less frequently in a population than would be expected from a random formation of haplotypes from alleles based on their frequencies. Linkage disequilibrium is typically caused by fitness interactions between genes or by such non-adaptive processes as population structure, inbreeding, and stochastic effects. In population genetics, linkage disequilibrium is said to characterize the haplotype distribution at two or more loci.

The term “genotype” as used herein refers to the identity of the alleles present in an individual or a sample. In the context of the present invention a genotype preferably refers to the description of the polymorphic alleles present in an individual or a sample. The term “genotyping” a sample or an individual for a polymorphic marker refers to determining the specific allele or the specific nucleotide sequence carried by an individual at a polymorphic marker.

### PREFERRED MODES FOR CARRYING OUT THE INVENTION

The present invention is directed to methods and kits for predicting the resistance (protection) of individuals to develop AIP.

The method of the invention for assessing resistance of a subject to develop antipsychotic-induced parkinsonism, comprises the following steps:
(a) obtaining a sample comprising genetic material from the subject;
(b) determining in said genetic material the presence of the nucleotide sequence of the ZFPM2 gene or a fragment thereof; and
(c) identifying in said nucleotide sequence the polymorphic site rs12678719,

wherein the presence of cytosine at rs12678719 is indicative of resistance to emergence or aggravation of antipsychotic-induced parkinsonism.

The method of the invention may further comprise the following additional steps:
(d) determining in said genetic material the presence of the nucleotide sequence of the RGS2 gene or a fragment thereof; and
(e) identifying in said nucleotide sequence the polymorphic site rs4606, wherein the presence of guanine at rs4606 is indicative of resistance to emergence or aggravation of antipsychotic-induced parkinsonism.

Thus, the method of the invention provides a platform for determining resistance to the development of EPS in three groups of subjects:
(i) carriers of the c allele in the rs12678719 SNP;
(ii) carriers of the g allele in the rs4606 SNP;
(iii) carriers of the c allele in the rs12678719 SNP and g allele in the rs4606 SNP;

Based on the principles of the present invention, the last group of subjects, namely, carriers of the g allele in the rs12678719 SNP and c allele in the rs4606 SNP are susceptible to development of extrapyramidal symptoms, such as AIP, upon treatment with typical antipsychotics.

Antipsychotic-induced parkinsonism (AIP) is a severe adverse affect of neuroleptic treatment. Clinically, AIP is very similar to idiopathic Parkinson's disease (PD). It is characterized by bradykinesia, tremor, rigidity, and stooped posture. Other manifestations are gait disturbance, salivation, and seborrheic dermatitis. AIP is thought to be caused by blockade of dopamine receptors in the nigrostriatal pathway, although additional hypotheses have been suggested. It has been shown that early EPS, including parkinsonism, are predictors of tardive dyskinesia, but the effect of EPS on antipsychotic treatment outcome is not clear.

The terms “antipsychotic(s)”, “typical antipsychotic(s)” and “conventional antipsychotic(s)” are interchangeably used herein to describe the first generation of antipsychotic medications used to treat psychosis (in particular, schizophrenia). Typical antipsychotics may also be used for the treatment of acute mania, agitation, and other conditions. Typical antipsychotics include haloperidol, penfluridol, sulpiride, zuclopenthixol, flupenthixol, clotiapine and phenothiazines, such as chlorpromazine, prochlorperazine, flupenazine, trifluoperazine, perphenazine, levomepromazine and thioridazine. These drugs cause serious side effects, the most common of which are extrapyramidal symptoms (EPS), particularly, dystonia (abnormal tonicity of the muscles), Parkinsonism and akathisia (motor restlessness), therefore antipsychotics are generally being replaced by atypical antipsychotic drugs.

Typical antipsychotic drugs, also called first generation or traditional antipsychotics, and atypical antipsychotic drugs (also called second generation antipsychotics) are indispensable in the pharmacological treatment of psychoses, such as schizophrenia and other neuropsychiatric conditions that are associated with psychotic states. EPS may develop within hours to days of the implementation of treatment. Longer-term treatment is associated with development of the chronic, choreoathetotic movement disorder, tardive dyskinesia. The unpleasant side effects induced by antipsychotics often lead patients to stop using them.

Atypical antipsychotics refer to a class of medications used to treat psychiatric conditions with more favorable side effect profile than typical antipsychotics with regard to induction of extrapyramidal symptoms. Due to the decreased propensity of atypical antipsychotics to cause extrapyramidal side effects and an absence of sustained prolactin elevation, atypical antipsychotics are now considered to be first line treatments for schizophrenia and are gradually replacing the typical antipsychotics. Atypical antipsychotics include, but are not limited to: Olanzapine, disclosed in U.S. Pat. No. 5,229,382; Clozapine, disclosed in U.S. Pat. No. 3,539,573; Risperidone, disclosed in U.S. Pat. No. 4,804,663; Sertindole, disclosed in U.S. Pat. Nos. 4,710,500; 5,112,838 and 5,238,945; Quetiapine, disclosed in U.S. Pat. Nos. 4,879,288; and Ziprasidone, typically administered as the hydrochloride monohydrate. Ziprasidone is disclosed in U.S. Pat. Nos. 4,831,031 and 5,312,925. Its utility in the treatment of schizophrenia is described in U.S. Pat. No. 4,831,031. Aripiprazole and a pharmaceutical solution comprising same are disclosed in U.S. Pat. Nos. 5,006,528 and 6,977,257, respectively.

Atypical, second-generation antipsychotics (SGAs) are generally considered less likely to cause EPS than typical, first-generation drugs (FGA), although EPS risk is not negligible with SGA. However, the Clinical Antipsychotic Trials of Intervention Effectiveness (CATIE) did not show a difference between SGA and the typical antipsychotic, perphenazine, with regard to acute EPS prevalence (Lieberman et al., N Engl J Med 353(12):1209-1223, 2005).

AIP prevalence data vary widely among studies, ranging from 15% to more than 50% of antipsychotic-treated patients. The substantial heterogeneity may stem from interstudy differences in medication regimens, patient demographic background data, and variable phenotype definitions.

According to well-documented clinical and demographic data, the major risk factors for developing AIP are: use of high-potency neuroleptics, old age, and female gender.

Approaches for assessing EPS prevalence, but not specifically AIP prevalence, known to date include an assay for predicting the potential ability of a drug to cause EPS in rats, as disclosed in U.S. Pat. No. 4,086,350. The assay is based on calculating the ratio of the drug's ED₅₀ (i.p.) for antagonism of amphetamine-induced rotation to the drug's ED₅₀ (i.p.) for blockade of shock avoidance acquisition. This assay is suitable for application in laboratory animals.

A method for reversing or preventing extrapyramidal side effects in a human due to neuroleptic treatment is disclosed in U.S. Pat. No. 5,137,712. The method comprises concurrent administration of the neuroleptic with S-adenosyl-L-methionine.

However, the aforementioned methods do not teach or suggest identifying susceptibility to AIP upon treatment with antipsychotic drug.

In recent years, genetic alterations, also termed DNA polymorphisms or markers including SNPs or combinations thereof, i.e. haplotypes, which cause or contribute to various diseases have been identified. Use of SNPs and haplotypes for identifying the likelihood to develop a particular disease was previously exemplified. For example, particular haplotypes within the BRCA1 gene, which indicate susceptibility to the pathology associated with breast, ovarian, prostate and other cancers are disclosed in U.S. Pat. No. 6,951,721. In another example, in one of the genes associated with Alzheimer's disease, apolipoprotein E or ApoE, SNPs affect disease development. This gene contains two SNPs that result in three possible alleles for this gene each allele differs by one DNA base, and the protein product of each gene differs by one amino acid. Research has shown that an individual who inherits at least one of the alleles will have a greater chance of getting Alzheimer's disease. Apparently, the change of one amino acid in the protein alters its structure and function enough to make disease development more likely.

A method for predicting the susceptibility or resistance of individuals to develop EPS during treatment with antipsychotic drugs, by identifying the presence of specific SNPs and haplotypes within the RGS2 gene, is disclosed in WO 2007/144874, by the inventors of the present invention, the contents of which is incorporated herein by reference in its entirety. Susceptibility or resistance of individuals to develop EPS during treatment with antipsychotic drugs was found to be associated with at least one polymorphic site in the RGS2 gene selected from the group consisting of: rs2179652, rs1933695, rs2746073, rs4606, rs1819741 and rs1152746.

According to data from the 1960s which relate to first generation drugs, 50% of cases manifest AIP within the first month of drug administration and 90% during the first 72 days. It was also shown that that majority of patients develop AIP within 20 days or even within the first week of treatment although improvement and recovery of AIP symptoms within 2 months was reported in two thirds of patients. However, AIP is also observed as a late-onset manifestation.

In addition to the epidemiological risk factors, genetic factors may contribute to interindividual differences in AIP susceptibility. Using the candidate gene approach, several polymorphisms within genes encoding receptors for dopamine and serotonin have been studied for association with AIP, but findings were not conclusive (Al Hadithy et al., Am. J. Med. Genet. B. Neuropsychiatr., Genet. 147B (6):890-897, 2008). Associations have been reported for the VNTR polymorphism in DATI, Taq1, and 141C1ns/Del variants in DRD2 (Al Hadithy et al.; ibid), and the HTR2c polymorphism Cys23Ser (Gunes et al., Eur. J. Clin. Pharmacol., 64(5):477-482, 2008). Genes associated with idiopathic Parkinson's disease such as Alpha-synuclein, LRRK2, Parkin, Pinkl, DJ-1, and UCHL1 have not been specifically studied for association with AIP thus far.

Genome-wide association studies (GWASs) are a well-established tool in the search for common genetic variations in complex disorders including psychiatric and neurological diseases. Several pharmacogenetic GWASs have been published recently, some of them with impressive success (review by Crowley et al., Pharmacogenomics 10(2):161-163, 2009). In contrast to candidate gene-based methods, the genome-wide pharmacogenomic approach allows unbiased, “hypothesis-free” detection of DNA variants associated with the phenotype of interest.

The present invention is based on the first case-control, pharmacogenomic GWAS for AIP severity and employs phenotype and genotype data from the CATIE project (Lieberman et al.; ibid). It further relies on a secondary analysis of the data that aimed to identify genetic variants associated with AIP severity. An analysis of 397 schizophrenia patients treated for at least 2 weeks with one antipsychotic drug and assessed regularly for AIP led to the discovery of polymorphic sites indicative of the tendency to develop AIP following treatment with antipsychotic drugs.

A person skilled in the art of psychiatry will find the present invention useful for planning an adequate treatment regimen for treating psychosis. Although it is known in the art that one of the most common and acute side-effect of antipsychotic treatments is AIP, to date, resistance or susceptibility to induction or enhancement of AIP cannot be assessed with high certainty and therefore is not a valid criterion for selecting a treatment regimen. The need to determine resistance to AIP is crucial since AIP often leads patients to stop using the medications. Moreover, AIP seriously damages patient's functioning and wellbeing.

It is to be understood that “resistance” or “protection” from IP or “susceptibility” to AIP as used herein do not necessarily mean that the subject will be resistant to AIP or will develop AIP, upon treatment with antipsychotic drugs but rather that the subject is, in a statistical sense, more likely to be resistant to AIP or to develop AIP than an average member of the population. As used herein, “resistance” or “susceptibility” to AIP induced by antipsychotic drugs may exist if the subject has one or more genetic determinants (e.g., polymorphic variants or alleles) that may, either alone or in combination with one or more other genetic determinants, contribute to an increased resistance to AIP or an increased risk of developing AIP in some or all subjects. Ascertaining whether a subject has any such genetic determinants according to the teaching of the present invention is useful, for example, for purposes of genetic counseling and for diagnostics tests before determining the treatment regimen of psychotic patients.

It is noted that the dichotomized phenotype established in the present invention was defined on the basis of the average of SAS mean global score measurements during CATIE phase 1 (not including baseline measurement), using an extreme distribution of phenotype-analysis approach. However, use of the average score of multiple clinical measurements of SAS-MGS during the phase 1 time period rather than a single measurement (e.g., the highest score) to determine individual AIP score is in keeping with the prospective nature of the CATIE study in which patients were followed for up to 18 months. Average scores are less prone to bias due to occasional outlying scores that may result from inter-individual differences in AIP evaluation, exceptional increases in drug doses, and changes in patient adherence to treatment during follow-up. Moreover, since AIP development is dose dependent and all patients are expected to eventually develop AIP if high-enough doses are prescribed (Hirose 2006), it is believed that relying on average SAS-MGS measurements taken over several months of follow-up is an appropriate strategy.

The analysis disclosed herein used the “best responders” (who did not develop any sign of AIP during the follow-up despite chronic treatment with antipsychotics) as controls while patients with the highest SAS-MGS scores (0.3 as a cutoff) were defined as cases. Focusing on the extremes of a sample distribution is regarded as one of the most advantageous strategies in conducting pharmacogenomic GWASs (Crowley et al.; ibid). To ensure that differences in individual SAS at baseline would not affect AIP scores during the study (the majority of patients were treated with antipsychotics before entering the study), this covariate in the logistic regression model was controlled.

Methodological limitations of this GWAS for AIP severity include the fact that five different antipsychotic drugs were prescribed, each with a different propensity to induce AIP (one FGA and four SGA). In addition, the doses were not uniform but adjusted individually. Thus, one may argue that AIP severity differences could stem from difference in drug allocation and/or higher doses between the case and control groups rather than genetic predisposition. However, in agreement with the findings of the original CATIE report (Lieberman et al.; ibid), association of drug type or average dose (standardized to chlorpromazine unit) during phase 1 with AIP severity was not observed (see Table 1). On the other hand, there was a statistically significant difference between cases and controls in concomitant use of anticholinergic medication (see Table 1). To overcome this possible confounder, the concomitant use of anticholinergic agents during phase 1 was included as a dichotomous covariate in the logistic regression model. A further point to be noted is that there are more males than females in the AIP group; this is contradictory to textbook knowledge that females are more susceptible to parkinsonism induced by antipsychotic drugs. However, in the overall CATIE sample, 74% of the participants available for genotyping were men; therefore, the core sample was not representative in terms of gender distribution.

In the present invention, AIP was assessed using the modified SAS. The original SAS is a ten-item scale commonly used to assess AIP in both research and clinical contexts but this scale has been criticized for over-emphasizing rigidity items as well as for differences in sensitivity between SAS and DSM-IV case definitions of neuroleptic-induced parkinsonism (Janno et al. 2004). In the CATIE study, a modified version of SAS was used (including six items). Although the number of items in the present SAS version is six instead of ten (as in the original version), the widely accepted SAS mean global score of 0.3 was used as a cutoff point for the existence of parkinsonism since this score reflects a mean and not a total score. In addition, and in accordance with the present “extremes of distribution” approach, the threshold of 0.3 and above approximately represents the upper third of the CATIE phase 1 average SAS-MGS while 0 approximately represents the lower third of the sample.

The number of pharmacogenomic GWASs reported in the literature, studying association with drug-induced phenotypes, is gradually increasing. These studies have a relatively small sample size compared with disease-oriented GWASs (which include thousands of participants). In spite of this limitation, some important and impressive pharmacogenomic findings have been reported mainly concerning relatively rare phenotypes. With relatively small sample sizes, it is easier to find susceptibility variants for rare side effects (resembling monogenetic heritability) than variants associated with common drug-induced phenotypes (Crowley et al.; ibid).

The present invention discloses several candidate genes that affect the tendency to develop AIP in the course of medical treatment with antipsychotic drugs, these include the gene EBF1⁻ (rs891903, intron 6, R=4.06×10⁻⁵) which encodes a transcription factor that controls neurogenesis in the CNS and is implicated in the development of nigrostriatal neurons. EBF1 also plays a regulatory role in the development of dopaminergic neurons and is critical for the migration of mesodiencephalic dopaminergic neurons to the substantia nigra. The RAPGEF5 gene was also discovered by the present invention as affecting the tendency to develop AIP in the course of medical treatment with antipsychotic drugs (rs7804311, intron c) P=5.64×10⁻⁵) and it is also known as MR-GEF, encodes a guanine nucleotide exchange factor which takes part in signaling pathways related to telencephalic neurogenesis.

Most of the top SNPs provided by the present invention are intergenic rather than located within annotated genes. Their distance to the nearest gene ranges from 177 base pairs (bp) to more than a million kb (see Table 4). For example, a top AIP severity-associated SNP (rs12476047) is located 146 kb away from the FIGN gene, which encodes the fidgetin protein, a member of the AAA family of ATPase that functions as a chaperone. This gene is involved in developmental processes in several body organs. Intergenic variants may play an important role in regulation of nearby gene expression, as enhancers, repressors, or transcription-factor binding sites. Moreover, the importance and prevalence of intergenic transcription, extensive transcription of non protein coding DNA regions outside annotated genes that may have regulatory role is recently being appreciated.

Another gene identified as associated with AIP severity is NOVA1; two intergenic AIP severity-associated SNPs, rs8006700 and rs1950420, are respectively located 95 and 74 kb away from the gene (Table 4). NOVA1 encodes a neuronal specific RNA-binding protein, which serves as an antigen recognized by the antisera of patients with the rare paraneoplastic opsoclonus-myoclonus ataxia (POMA). POMA affects motor neurons in the brain stem, cerebellum, and spinal cord, and is associated with several types of cancer.

The underlying pathophysiology of AIP is unclear but, but without being bound to any theory or mechanism, AIP is probably mediated by decreased dopaminergic transmission along the nigrostriatal pathway. It is well established that dopamine D2 receptor occupancy by antipsychotics in the nigrostriatal pathway is related to parkinsonism, and all the clinically effective antipsychotics drugs block this receptor. Occupancy of more than 80% of D2 receptors by typical antipsychotics substantially increases the risk of AIP while atypical antipsychotic D2 receptor occupancy is usually lower and depends on the specific drug. Other hypotheses of AIP mechanisms focus on differences in the dissociation rate of typical versus atypical drugs from D2 receptors and/or the contribution of serotonin receptors blockade. Thus, genetic variants may influence susceptibility to AIP by more than one biological mechanism.

Although it is not clear whether EPS are associated with poorer or better pharmacological treatment outcomes, it is possible that susceptibility genes for AIP are risk factors for a heritable schizophrenia endophenotype reflecting a dopaminergic disturbance in the basal ganglia. Thus, the top AIP candidate genes provided by the present invention could also relate to the genetics of schizophrenia or idiopathic PD.

The implications of EPS, and AIP in particular, for the quality of daily life of antipsychotic-treated patients may become substantial. AIP can result in weakness, muscle aching, impaired ability to perform occupational and social tasks due to impaired dexterity, patients can suffer from social stigma and distress and, in severe cases, lead to falls and injury. Thus, a priori prediction of AIP susceptibility is an important clinical need for better management of vulnerable patients, maintaining low drug doses, early treatment with anticholinergic agents, and preference for SGA. In addition, indentifying susceptibility or protective genetic variants associated with AIP contribute to the basic understanding of pathophysiology underlying AIP.

It should be understood that diagnosing resistance to AIP or predisposition to AIP by detecting variant mRNAs or the gene product(s) disclosed herein, are also encompassed within the scope of the present invention. As used herein a “variant mRNA” or a “variant gene product” refer to a mRNA or a gene product, respectively, which are spliced or encoded by the variant allele comprising at least one polymorphic site according to the present invention, including, but not limited to, a full length mRNA or gene product, an essentially full-length mRNA or gene product and a biologically active fragment of the gene product. Biologically active fragments include any portion of the full-length polypeptide which confers a biological function on the variant gene product, including ligand binding and antibody binding. Ligand binding includes binding by nucleic acids, proteins or polypeptides, small biologically active molecules, or large cellular structures.

A variant gene product is also intended to mean gene products which have altered expression levels or expression patterns which are caused, for example, by the variant allele of regulatory sequence(s).

DNA, as analyzed herein for determining the presence of SNPs within genes or fragments thereof in a subject treated with antipsychotic drugs, may be extracted from virtually any body sample, such as blood (other than pure red blood cells), tissue material and the like by a variety of techniques such as that described by Maniatis, et. al. (Molecular Cloning: A Laboratory Manual, Cold Spring Harbor, N.Y., pp. 280-281, 1982). Convenient tissue samples include whole blood, semen, saliva, tears, urine, fecal material, sweat, buccal material, skin and hair. For assay of cDNA or mRNA, the tissue sample must be obtained from an organ in which the target nucleic acid is expressed. According to certain embodiments, the genomic DNA sample is obtained from whole blood samples or EBV-transformed lymphoblast lines. The sample can be obtained from any suitable subject, i.e. an adult, child, fetus, or embryo. According to certain embodiments of the invention the sample is obtained prenatally, either from the fetus or embryo or from the mother (e.g., from fetal or embryonic cells that enter the maternal circulation).

Typically, the sample obtained from the subject is processed before the detecting step, e.g. the DNA in the cell or tissue is separated from other components of the sample, and the target DNA is amplified as described herein below. All samples obtained from a subject, including those subjected to any sort of further processing, are considered to be obtained from the subject.

If the extracted sample is impure, it may be treated before analysis with an amount of a reagent effective to open the cell membranes of the sample, and to expose and/or separate the strand(s) of the nucleic acid(s). This lysing and nucleic acid denaturing step exposes and separates the strands.

The methods and kits of the invention are directed to assessment of susceptibility or resistance to the development of EPS, including AIP, in patients treated or intended to be treated with one or more antipsychotic drugs, by identifying the presence of the SNP rs12678719 in the ZPFM2 gene, alone or in combination with the SNP rs4606 in the RGS2 gene, within a bodily sample taken from the patients.

According to the present invention the phenotype that is resistant to antipsychotics induced EPS, such as, IP, is the phenotype having the ‘c’ allele of rs12678719 in the ZFPM2 gene. An exemplary DNA sequence corresponding to this phenotype includes cytosine at rs12678719 and is set for the in SEQ ID NO: 1 (the polymorphic site is underlined):

ATAGGAAAAA AGACTGGCCT GGCTCTATGC ATTGGGAACA GACTCCAAAA TATCAGTGAG
CCACACTTGA TATCTGTGAG CAGGGTTATG TGATATTATT AATGAACTAA CCAGAGTGTG
CCATGAAAGC TTAACATACT TTAAAGTATT TGTAGAGAGA AAATAATAAA CACTATTAGC
ACATAGTTCC AAGCACCCTT AAGGAAGGTA CTATTTACTC ACATTGATAA AGTCTTATTT
AAGCTATTTG ACTAGAAAAG GTATGTGGTA GAGGAGGAAA AGTGTTTTTA TTATAACTGA
CCCTAACAATG ACCTAGAAAA CAATTTGAAA TGATTGAATG ATGAACTTGA TTCCTTGGCT
TGCAGTAGAA GAATTTTAAG GTTTATTAAT CACTGCAGTC GCTGCTGGTA TCCATTCTGT
TTTGTGGCAT CATTTGAACC CCATGATTTC ATGAATAACA TTCCCCTGCA GATAGTTGGT
TGGAGCTGCC CTTATTTTTT AAATGTTTGT ATTCTGTGGC TTGTCAAGAA GTAAGGAACA
GGGCTTTCTT AGAGATAACT CCCCTAGGAT TAAATCCAAG GGAGTGGTTC TTAAACATTT

Another phenotype that is resistant to antipsychotics induced EPS, such as, IP, is the phenotype having the ‘g’ allele of rs4606 in the RGS2 gene. An exemplary DNA sequence corresponding to this phenotype includes guanine at rs4606 and is set for the in SEQ ID NO: 4 (the polymorphic site is underlined):

CTATGTGCAAGGGTATTGAAG

The phenotype that is susceptible to antipsychotics induced EPS, such as, IP, is the phenotype having the ‘g’ allele of rs12678719 in the ZFPM2 gene and the ‘c’ allele of rs4606 in the RGS2 gene. Exemplary DNA sequences corresponding to these phenotypes include guanine at rs12678719, as set forth for example by SEQ ID NO: 2, (the polymorphic site is underlined):

ATAGGAAAAA AGACTGGCCT GGCTCTATGC ATTGGGAACA GACTCCAAAA TATCAGTGAG
CCACACTTGA TATCTGTGAG CAGGGTTATG TGATATTATT AATGAACTAA CCAGAGTGTG
CCATGAAAGC TTAACATACT TTAAAGTATT TGTAGAGAGA AAATAATAAA CACTATTAGC
ACATAGTTCC AAGCACCCTT AAGGAAGGTA CTATTTACTC ACATTGATAA AGTCTTATTT
AAGCTATTTG ACTAGAAAAG GTATGTGGTA GAGGAGGAAA AGTGTTTTTA TTATAACTGA
GCCTAACAATG ACCTAGAAAA CAATTTGAAA TGATTGAATG ATGAACTTGA TTCCTTGGCT
TGCAGTAGAA GAATTTTAAG GTTTATTAAT CACTGCAGTC GCTGCTGGTA TCCATTCTGT
TTTGTGGCAT CATTTGAACC CCATGATTTC ATGAATAACA TTCCCCTGCA GATAGTTGGT
TGGAGCTGCC CTTATTTTTT AAATGTTTGT ATTCTGTGGC TTGTCAAGAA GTAAGGAACA
GGGCTTTCTT AGAGATAACT CCCCTAGGAT TAAATCCAAG GGAGTGGTTC TTAAACATTT

AND

cytosine at rs4606, as set forth for example by SEQ ID NO: 3 (the polymorphic site is underlined): CTATGTGCAACGGTATTGAAG

RGS and RGS-like proteins are a family of more than 30 members, defined by a common RGS domain, responsible for G-alpha binding, stimulating GTPase activity and termination of downstream signals. In animal models, RGS protein expression is influenced by administration of antipsychotic drugs. RGS2 influences the D1 receptor pathway and dopamine receptors agonists and antagonists may regulate the expression of RGS2 and RGS4.

In the human context, some of the RGS genes including RGS2 have been shown to be expressed in brain. RGS2 is a small gene (3,235 bp). Two of the 5 SNPs that are considered herein are located within the gene (rs2746073 is intronic and rs4606 is in the 3′ UTR).

SNPs in the RGS2 gene, including inter alia rs2746073 and rs4606, and haplotypes comprising same were shown to be associated with several anxiety disorders phenotypes. The presence of a particular human D2 receptor gene allele was found to correlate with susceptibility to compulsive disorder, as disclosed in U.S. Pat. No. 5,500,343.

The zinc finger protein encoded by the ZFPM2 gene is a widely expressed member of the FOG family of transcription factors. The family members modulate the activity of GATA family proteins, which are important regulators of hematopoiesis and cardiogenesis in mammals. It has been demonstrated that the protein can both activate and down-regulate expression of GATA-target genes, suggesting different modulation in different promoter contexts. A related mRNA suggests an alternatively spliced product.

The DNA obtained from a subject, for determining the presence of polymorphisms in the genes examined is typically amplified. The deoxyribonucleotide triphosphates dATP, dCTP, dGTP, and dTTP are added to the synthesis mixture, either separately or together with the primers, in adequate amounts and the resulting solution is heated. After the heating period, the solution is allowed to cool, which is preferable for the primer hybridization. To the cooled mixture is added an appropriate agent for effecting the primer extension reaction (called herein “agent for polymerization”), and the reaction is allowed to occur under conditions known in the art. The agent for polymerization may also be added together with the other reagents if it is heat stable. This synthesis (or amplification) reaction may occur at room temperature up to a temperature above which the agent for polymerization no longer functions. Thus, for example, if DNA polymerase is used as the agent, the temperature is generally no greater than about 40° C. Most conveniently the reaction occurs at room temperature. The primers used to amplify the strands corresponding to RGS2 gene or fragments thereof are oligonucleotides of sufficient length and appropriate sequence to provide initiation of polymerization. Environmental conditions conducive to synthesis include the presence of nucleoside triphosphates and an agent for polymerization, such as DNA polymerase, and a suitable temperature and pH. Each primer is preferably single stranded for maximum efficiency in amplification, but may be double stranded. If double stranded, the primer is first treated to separate its strands before being used to prepare extension products. The primer must be sufficiently long to prime the synthesis of extension products in the presence of the inducing agent for polymerization. The exact length of primer will depend on many factors, including temperature, buffer, and nucleotide composition. The oligonucleotide primer typically contains 12-20 or more nucleotides, although it may contain fewer nucleotides.

The term “primer” refers to a single-stranded oligonucleotide capable of acting as a point of initiation of template-directed DNA synthesis under appropriate conditions (i.e., in the presence of four different nucleoside triphosphates and an agent for polymerization, such as, DNA or RNA polymerase or reverse transcriptase) in an appropriate buffer and at a suitable temperature. The appropriate length of a primer depends on the intended use of the primer but typically ranges from 15 to 30 nucleotides. Short primer molecules generally require cooler temperatures to form sufficiently stable hybrid complexes with the template. A primer need not reflect the exact sequence of the template but must be sufficiently complementary to hybridize with a template. The term primer site refers to the area of the target DNA to which a primer hybridizes. The term primer pair means a set of primers including a 5′ upstream primer that hybridizes with the 5′ end of the DNA sequence to be amplified and a 3′, downstream primer that hybridizes with the complement of the 3′ end of the sequence to be amplified.

Primers used to carry out this invention are designed to be substantially complementary to each strand of the genomic locus to be amplified. This means that the primers must be sufficiently complementary to hybridize with their respective strands under conditions which allow the agent for polymerization to perform. In other words, the primers should have sufficient complementarity with the 5′ and 3′ sequences flanking the mutation to hybridize therewith and permit amplification of the genomic locus.

The oligonucleotide primers of the invention may be prepared using any suitable method, such as conventional phosphotriester and phosphodiester methods of automated embodiments thereof. In one such automated embodiment, diethylphosphoramidites are used as starting materials and may be synthesized as described by Beaucage, et al., (Tetrahedron Letters, 1981; 22:1859-1862). Alternatively, the primers of the invention may be synthesized on a modified solid support as described in U.S. Pat. No. 4,458,066.

The agent for polymerization may be any compound or system which will function to accomplish the synthesis of primer extension products, including enzymes. Suitable enzymes for this purpose include, for example, E. coli DNA polymerase I, Klenow fragment of E. coli DNA polymerase, polymerase muteins, reverse transcriptase, other enzymes, including heat-stable enzymes (i.e., those enzymes which perform primer extension after being subjected to temperatures sufficiently elevated to cause denaturation), such as Taq polymerase. Suitable enzyme will facilitate combination of the nucleotides in the proper manner to form the primer extension products which are complementary to each polymorphic locus nucleic acid strand. Generally, the synthesis will be initiated at the 3′ end of each primer and proceed in the 5′ direction along the template strand, until synthesis terminates, producing molecules of different lengths.

The newly synthesized strand and its complementary nucleic acid strand will form a double-stranded molecule under hybridizing conditions described above and this hybrid is used in subsequent steps of the process. In the next step, the newly synthesized double-stranded molecule is subjected to denaturing conditions using any of the procedures described above to provide single-stranded molecules. The steps of denaturing, annealing, and extension product synthesis can be repeated as often as needed to amplify the target polymorphic locus nucleic acid sequence to the extent necessary for detection. The amount of the specific nucleic acid sequence produced will accumulate in an exponential fashion. Amplification is described in PCR—A Practical Approach, ILR Press, Eds. McPherson, Quirke and Taylor, 1992.

Although the method of amplifying is preferably PCR, as described herein and as is commonly used by those of ordinary skill in the art, alternative methods of amplification can also be employed as long as the genetic locus amplified by PCR using primers of the invention is similarly amplified by the alternative means. Such alternative amplification systems include but are not limited to self-sustained sequence replication, which begins with a short sequence of RNA of interest and a T7 promoter. Reverse transcriptase copies the RNA into cDNA and degrades the RNA, followed by reverse transcriptase polymerizing a second strand of DNA. Another nucleic acid amplification technique is nucleic acid sequence-based amplification (NASBA) which uses reverse transcription and T7 RNA polymerase and incorporates two primers to target its cycling scheme. NASBA can begin with either DNA or RNA and finish with either, and amplifies to 10⁸ copies within 60 to 90 minutes. Alternatively, nucleic acid can be amplified by ligation activated transcription (LAT). LAT works from a single-stranded template with a single primer that is partially single-stranded and partially double-stranded. Amplification is initiated by ligating a cDNA to the promoter oligonucleotide and within a few hours, amplification is 10⁸ to 10⁹ fold. Another amplification system useful in the method of the invention is the QB Replicase System. The QB replicase system can be utilized by attaching an RNA sequence called MDV-1 to RNA complementary to a DNA sequence of interest. Another nucleic acid amplification technique, ligase chain reaction (LCR), works by using two differently labeled halves of a sequence of interest which are covalently bonded by ligase in the presence of the contiguous sequence in a sample, forming a new target. The repair chain reaction (RCR) nucleic acid amplification technique uses two complementary and target-specific oligonucleotide probe pairs, thermostable polymerase and ligase, and DNA nucleotides to geometrically amplify targeted sequences. A 2-base gap separates the oligonucleotide probe pairs, and the RCR fills and joins the gap, mimicking DNA repair. Nucleic acid amplification by strand displacement activation (SDA) utilizes a short primer containing a recognition site for HincII with short overhang on the 5′ end which binds to target DNA. A DNA polymerase fills in the part of the primer opposite the overhang with sulfur-containing adenine analogs. HincII is added but only cuts the unmodified DNA strand. A DNA polymerase that lacks 5′ exonuclease activity enters at the site of the nick and begins to polymerize, displacing the initial primer strand downstream and building a new one which serves as more primer. SDA produces greater than 10⁷-fold amplification in 2 hours at 37° C. Unlike PCR and LCR, SDA does not require instrumented temperature cycling. Another method is a process for amplifying nucleic acid sequences from a DNA or RNA template which may be purified or may exist in a mixture of nucleic acids. The resulting nucleic acid sequences may be exact copies of the template, or may be modified. The process has advantages over PCR in that it increases the fidelity of copying a specific nucleic acid sequence, and it allows one to more efficiently detect a particular point mutation in a single assay. A target nucleic acid is amplified enzymatically while avoiding strand displacement. Three primers are used. A first primer is complementary to the first end of the target. A second primer is complementary to the second end of the target. A third primer which is similar to the first end of the target and which is substantially complementary to at least a portion of the first primer such that when the third primer is hybridized to the first primer, the position of the third primer complementary to the base at the 5′ end of the first primer contains a modification which substantially avoids strand displacement. This method is detailed in U.S. Pat. No. 5,593,840. Although PCR is the preferred method of amplification if the invention, these other methods can also be used to amplify the gene of interest.

The amplification products may be detected by Southern blots analysis, without using radioactive probes. In such a process, for example, a small sample of DNA containing a very low level of the nucleic acid sequence of the polymorphic locus is amplified, and analyzed via a Southern blotting technique or similarly, using dot blot analysis. The use of non-radioactive probes or labels is facilitated by the high level of the amplified signal. Alternatively, probes used to detect the amplified products can be directly or indirectly detectably labeled, for example, with a radioisotope, a fluorescent compound, a bioluminescent compound, a chemiluminescent compound, a metal chelator or an enzyme. Those of ordinary skill in the art will know of other suitable labels for binding to the probe, or will be able to ascertain such, using routine experimentation.

Sequences amplified by the methods of the invention can be further evaluated, detected, cloned, sequenced, and the like, either in solution or after binding to a solid support, by any method usually applied to the detection of a specific DNA sequence such as PCR, oligomer restriction (Saiki, et al., Bio/Technology, 1985; 3:1008-1012), allele-specific oligonucleotide (ASO) probe analysis (Conner, et al., Proc. Natl. Acad. Sci. U.S.A., 1983; 80:278), oligonucleotide ligation assays (OLAs; Landgren, et al., 1988; Science, 241:1007), heteroduplex analysis, chromatographic separation and the like. Molecular techniques for DNA analysis have been reviewed (Landgren, et al., Science, 1988; 242:229-237).

A number of methods well known in the art can be used to carry out the sequencing reactions. Commonly, enzymatic sequencing based on the Sanger dideoxy method is used as described, for example, in Sanger et al., Proc. Natl. Acad. Sci. 1977; 74:5463. Mass spectroscopy may also be used. Well known sequencing methods also include Maxam-Gilbert chemical degradation of DNA (see Maxam and Gilbert, Methods Enzymol., 1980; 65:499). One skilled in the art recognizes that sequencing is now often performed with the aid of automated methods.

The sequencing reactions can be analyzed using methods well known in the art, such as polyacrylamide gel electrophoresis. In a preferred embodiment for efficiently processing multiple samples, the sequencing reactions are carried out and analyzed using a fluorescent automated sequencing system such as the Applied Biosystems, Inc. (“ABI”, Foster City, Calif.) system. For example, PCR products serving as templates are fluorescently labeled using the Taq Dye Terminator™ Kit (Perkin-Elmer). Dideoxy DNA sequencing is performed in both forward and reverse directions on an ABI automated Model 3.77™ sequencer. The resulting data can be analyzed using “Sequence Navigator™” software available through ABI. Alternatively, large numbers of samples can be prepared for and analyzed by capillary electrophoresis, as described, for example, in U.S. Pat. No. 5,498,324.

Determining the presence and identity of SNPs or haplotypes which correlate with onset or increase in AIP during treatment with antipsychotic drugs may be carried out by any one of the various tools for the detection of polymorphism on a target DNA known in the art, including, but not limited to, allele-specific probes, allele specific primers, direct sequencing, denaturing gradient gel electrophoresis and single-strand conformation polymorphism. Preferred techniques for SNP genotyping should allow large scale automated analysis, which do not require extensive optimization for each SNP analyzed.

The phrase “identifying a polymorphism” or “identifying a polymorphic variant” as used herein generally refers to determining which of two or more polymorphic variants exists at a polymorphic site. In general, for a given polymorphism, any individual will exhibit either one or two possible variants at the polymorphic site (one on each chromosome). This may, however, not be the case if the individual exhibits one more chromosomal abnormality such as deletions.

Oligonucleotides that exhibit differential or selective binding to polymorphic sites may readily be designed by one of ordinary skill in the art. For example, an oligonucleotide that is perfectly complementary to a sequence that encompasses a polymorphic site (i.e., a sequence that includes the polymorphic site within it or at least at one end) will generally hybridize preferentially to a nucleic acid comprising that sequence as opposed to a nucleic acid comprising an alternate polymorphic variant.

The design and use of allele-specific probes for analyzing polymorphisms is described, for example, in U.S. Pat. No. 5,348,855 and International Application WO 89/11548. Allele-specific probes can be designed that hybridize to a segment of target DNA from one individual but do not hybridize to the corresponding segment from another individual due to the presence of different polymorphic forms in the respective segments from the two individuals. Hybridization conditions should be sufficiently stringent that there is a significant difference in hybridization intensity between alleles, and preferably an essentially binary response, whereby a probe hybridizes to only one of the alleles. Typically, a probe comprises a region of nucleotide sequence that hybridizes to at least about 8, preferably to about 10 to 15, more preferably to about 20-25 and most preferably to about 40-75 consecutive nucleotides of a nucleic acid molecule. Preferably, the probes are designed as to be sufficiently specific to be able to discriminate the targeted sequence for only one nucleotide variation. According to certain embodiments, the probes are labeled cl immobilized on a solid support by any suitable method as is known to a person skilled in the art. The probes can be used in Southern hybridization to genomic DNA or Northern hybridization to mRNA; the probes can also be used to detect PCR amplification products. By assaying the hybridization to an allele specific probe, one can detect the presence or absence of a polymorphism in a given sample. Allele-specific probes are often used in pairs, one member of a pair showing a perfect match to a reference form of a target sequence and the other member showing a perfect match to a variant form. Several pairs of probes can then be immobilized on the same support for simultaneous analysis of multiple polymorphisms within the same target sequence. High-Throughput parallel hybridizations in array format are particularly preferred to enable simultaneous analysis of a large number of samples.

Alternative method for the detection and identification of polymorphism on a target DNA utilizes allele-specific primers, as described herein above. The direct analysis of the sequence of polymorphisms of the present invention can be accomplished using either the dideoxy chain termination method or the Maxam Gilbert method (see Sambrook et al., Molecular Cloning, A Laboratory Manual (2nd Ed., CSHP, New York 1989); Zyskind et al., Recombinant DNA Laboratory Manual, (Acad. Press, 1988)). It should be recognized that the field of DNA sequencing has advanced considerably in the past several years, specifically in reliable methods of automated DNA sequencing and analysis. These advances and those to come are explicitly encompassed within the scope of the present invention. As is known to a person skilled in the art, an amplified product can be sequenced directly or subcloned into a vector prior to sequence analysis.

Alleles of target sequences can be differentiated using single-strand conformation polymorphism analysis, which identifies base differences by alteration in electrophoretic migration of single stranded PCR products. Amplified PCR products can be generated as described above, and heated or otherwise denatured, to form single stranded amplification products. Single-stranded nucleic acids may refold or form secondary structures which are partially dependent on the base sequence. The different electrophoretic mobility of single-stranded amplification products can be related to base-sequence difference between alleles of target sequences.

Another method for rapid and efficient SNP analysis makes use of thermal denaturation differences due to differences in DNA base composition. In one embodiment of this test, allele, specific primers are designed as above to detect biallelic SNP with the exception that a 5′ GC tail of 26 bases is added to one primer. After PCR amplification with a single, common reverse primer, a fluorescent dye that binds preferentially to dsDNA (e.g., SYBR Green 1) is added to the tube and then the thermal denaturation profile of the dsDNA product of PCR amplification is determined. Samples homozygous for the SNP amplified by the GC tailed primer will denature at the high end of the temperature scale, while samples homozygous for the SN amplified by the non-GC tagged primer will denature at the low end of the temperature scale. Heterozygous samples will show two peaks in the thermal denaturation profile.

The invention further contemplates modifications of the methods described above, including, but not limited to allele-specific hybridization on filters, allele-specific PCR, fluorescence allele-specific PCR, PCR plus restriction enzyme digest (RFLP-PCR), denaturing capillary electrophoresis, dynamic allele-specific hybridization (DASH), 5′ nuclease (Taq-Man™) assay, and the primer extension and time-of-flight mass spectrometry. According to certain currently preferred embodiments, the polymorphism of the present invention is detected using the primer extension and time-of-flight mass spectrometry method as exemplified herein below.

### EXAMPLES

### Example 1

### AIP Association Study of GWAS Findings

Sample description and clinical methods: The sample is essentially the same as reported by us in a previous study (Greenbaum et al, Pharmacogenomics J. 9:103-110, 2009) although slightly smaller in terms of participant number (DNAs of 6 patients were unavailable). In brief, this was a cross-sectional study of patients with schizophrenia or schizoaffective disorder diagnosed according to DSM-IV criteria who were hospitalized at one of three tertiary care public hospitals in the United States and had been treated with a single antipsychotic agent (clozapine, olanzapine, risperidone or a typical antipsychotic) for at least a month. Patients gave written informed consent for participation in the study after the purpose and procedures were explained. The protocol and consent forms were approved by the Internal Review Board of each institution. Recruitment was consecutive and sampling procedures were continued until there were approximately 50 patients in each of the 4 groups. Clinical state was evaluated by Positive and Negative symptoms scale (PANSS). AIP was evaluated by the Simpson Angus Scale (SAS). The scale was administered on two separate occasions, separated by at least a week, by the same clinician. The mean score of the two SAS assessments was used for data analysis. Data on evaluation of tardive dyskinesia and akathisia evaluation were also collected but were not analyzed in the current context.

The overall sample for the current study (clinical ratings and DNA available) consisted of 178 patients of whom 111 were African-American (AA) and 67 Caucasians (40 of Hispanic and 27 of European origin). Further demographic and clinical data, including distribution among the antipsychotic treatment groups and total PANSS score, is given in Table 1.

SNP selection and Genotyping: 15 SNPs, which were associated with AIP in our previous study (Alkelai et al, ibid) with a P value<0.0001 were selected for the current study. SNP genotyping was performed with the Sequenom MassARRAY system, at the Washington University Human Genetics Division Genotyping Core, St. Louis, USA. Quality control measures were implemented.

Phenotype definition: A dichotomized AIP severity phenotype was used, based on the average of the two SAS mean scores (SASms) rated for a particular patient during his research participation period. Since a SASms threshold of 0.3 for parkinsonism is commonly accepted and was also used in our previous AIP-GWAS (Alkelai et al, ibid), cases (AIP+) were defined as individuals whose average SASms was 0.3 and above, and controls (AIP−) as patients with average SASms less than 0.3.

For an additional analysis of extreme distribution of the phenotype, we defined controls as patients as those whose SASms was zero (absence of any parkinsonian features on two measurements), while cases were the same as described above (AIP+). This is a much more rigorous definition for controls, identical to the control definition in the AIP GWAS (Alkelai et al, ibid). However, only a small number of patients (32) met this extreme control criterion (Table 1).

Data analysis: To study association of the genotyped SNPs with AIP severity (dichotomized definition of the phenotype) in the US sample, we used logistic regression (additive model). Due to the mixed ethnicity of the US sample and its possible influence on allele frequencies, self reported ancestry (African Americans, White, Hispanic) was included as covariate. In addition, based on clinical considerations (see Introduction), we identified 4 potential covariates to be checked for inclusion in our regression model: gender; age; antipsychotic type; total PANSS score (indication for disease severity and therefore probably related to higher antipsychotics dose). To select covariates, we checked for association of these variables with the dependent variable (AIP+/AIP−) using T test or Chi-Square tests. Only significant variables (p<0.05) in the univariate analysis (conducted with SPSS Inc., Chicago, Ill., USA) were included in the regression model. The final analysis was performed using PLINK software. Hardy-Weinberg equilibrium for the studied SNPs was calculated with Haploview, version 4.1. The same analysis was implemented for the African-Americans subsample (without ancestry covariate).

Level of statistical significance required: We followed the criteria of Van der Oord et al. (Arch Gen Psychiatry, 65(9):1062-71, 2008) concerning the appropriate required significance level. According to these criteria, an uncorrected standard P value of <0.05 may be used in a replication trial, if association is for the same SNP and phenotype and the direction of effect is the same as in the original report.

The 15 selected SNPs (P<1×10⁻⁴ in the original AIP-GWAS) were successfully genotyped in the US AIP sample. One SNP (rs7174597) had a minor allele frequency<5%, and was therefore excluded. None of the remaining SNPs showed deviation from HWE. In the univariate analysis, we detected significant association of total PANSS score with AIP (extreme phenotype) in both overall and African-Americans subsamples. This variable, in addition to ethnicity, was included in the regression model as a covariate. There was no significant association of age, sex and antipsychotic type with AIP (wide and extreme phenotype definitions); thus they were not included in the regression model. Out of 14 analyzed SNPs, association of the ZFPM2 gene intronic SNP, rs12678719, with AIP (P=5.97×10⁻⁵ in the GWAS) was validated. Controlling for ethnicity and PANSS total score, the ‘G’ allele of this SNP was found to be a susceptibility allele (same direction as in the original report), when comparing AIP affected patients (AIP+, SASms>0.3) (N=62) to those who do not have AIP (AIP−, SASms<0.3) (N=116) (p=0.009; OR=1.93) (Tables 2 and 3).

Table 2 presents logistic regression for rs12678719 association with AIP severity, in the US sample (N=178). Table 3 exhibits logistic regression for rs12678719 association with AIP severity, in the African American subsample (N=111). In both tables, results for two definitions of controls (AIP+/AIP− and extremes) are shown, controlled for ethnicity and PANSS total score.

TABLE 1
Demographic and clinical description of the AIP US sample and the African-American
subsample.

| | Whole sample (N = 178) | | | | | | African American (N = 111) | | | | | |
|---|---|---|---|---|---|---|---|---|---|---|---|---|
| | | | | | Extreme | | | | | | Extreme | |
| | | | | | Controls | | | | Controls | | Controls | |
| | Cases (AIP+) | | Controls (AIP−) | | (SAS = 0) | | Cases (AIP+) | | (AIP−) | | (SAS = 0) | |
| Number | 62 | | 116 | | 41 | | 38 | | 73 | | 29 | |
| Age | 39.05 | (8.66) | 40.95 | (10.05) | 40.07 | (9.13) | 39.34 | (8.38) | 41.32 | (9.83) | 41.1 | (8.8) |
| Females | 2 | | 9 | | 2 | | 1 | | 5 | | 2 | |
| SAS mean score | 0.5 | (0.18) | 0.1 | (0.094) | 0 | | 0.53 | (0.19) | 0.09 | (0.09) | 0 | |
| PANSS total score | 70.45 | (15.48) | 67.17 | (17.47) | 59.77 | (11.83)* | 68.47 | (14.87) | 64.86 | (14.67) | 60 | (12.32)* |
| Antipsychotic | | | | | | | | | | | | |
| treatment: | | | | | | | | | | | | |
| Typical | 16 | (25.8) | 28 | (24.13) | 8 | (19.51) | 11 | (28.94) | 20 | (27.39) | 7 | (24.13) |
| Risperidone | 12 | (19.35) | 24 | (20.69) | 14 | (34.14) | 8 | (21.05) | 24 | (32.87) | 10 | (34.38) |
| Olanzapine | 19 | (30.64) | 28 | (24.13) | 12 | (29.26) | 10 | (26.31) | 16 | (21.91) | 7 | (24.13) |
| Clozapine | 15 | (24.2) | 26 | (22.41) | 7 | (17.07) | 9 | (23.68) | 13 | (17.8) | 5 | (17.24) |
| Ethnicity: | | | | | | | | | | | | |
| African Americans | 38 | (61.29) | 73 | (62.93) | 29 | (70.73) | | | | | | |
| Caucasians (including | 24 | (38.7) | 43 | (37.06) | 12 | (29.27) | | | | | | |
| Hispanic) | | | | | | | | | | | | |

Abbreviations:
SAS. Simpson Angus scale;
PANSS, Positive and Negative Symptoms scale.
*<0.05

TABLE 2
RS12678719 Association tests results in US sample

| | | | | | Controls | Alellic P | |
|---|---|---|---|---|---|---|---|
| risk | Cases | Controls | Alellic P | | (extreme) | value | |
| Allele | freq. | freq. | value | OR | frequency | (extremes) | OR |
| G | 0.603 | 0.474 | 0.009 | 1.93 | 0.475 | 0.017 | 2.19 |
| | | | | (1.18-3.13) | | | (1.15-4.2) |

TABLE 3
RS12678719 Association tests results in African American subsample

| | | | | | Controls | | |
|---|---|---|---|---|---|---|---|
| risk | Cases | Controls | Alellic P | | (extreme) | Alellic P value | |
| Allele | freq. | freq. | value | OR | freq. | (extremes) | OR |
| G | 0.75 | 0.528 | 0.002 | 2.85 | 0.57 | 0.06 | 2.19 |
| | | | | (1.47-5.81) | | | (0.98-4.85) |

When applying the same extreme phenotype definition used in the original GWAS, identifying controls as individuals with SASms of zero (N=41) and cases as individuals with SASms>0.3 (N=62), the association is still observed (p=0.017; OR=2.19) despite the decreased power. Since African-Americans represent the major ethnic group in this US sample, we studied the association of rs12678719 with AIP severity among AA separately. Association of the ‘G’ allele with AIP susceptibility was demonstrated, comparing AIP+ (N=38) to AIP− (N=73) (p=0.002; OR=2.85). None of the other SNPs studied reached statistically significance (Table 4).

TABLE 4
SNPs studied in the US AIP sample (AIP+/AIP−, N = 178). These SNPs were associated with AIP in the CATIE AIP-GWAS
at P value < 0.0001.

| | | | | | | | | | | Distance | US AIP |
|---|---|---|---|---|---|---|---|---|---|---|---|
| | | | Coordinate | | | | Minor | Odds ratio | Closest | to closest | sample |
| Annotation | P | Chr. | (annotated) | SNP type | Allele | MAF | allele | (95% CI) | gene | gene | P value |
| rs12476047 | 3.13E−06 | 2 | 164026122 | intergenic | c/t | 0.25 | c | 3.21 (1.97-5.25) | FIGN | 146242 | 0.65 |
| rs10818129 | 2.04E−05 | 9 | 118091021 | intergenic | g/a | 0.37 | g | 2.39 (1.6-3.57) | TLR4 | 1415260 | 0.31 |
| rs4725675 | 2.16E−05 | 7 | 139656374 | intergenic | c/t | 0.2 | c | 2.88 (1.77-4.69) | SLC37A3 | 23647 | 0.20 |
| rs8006700 | 2.31E−05 | 14 | 26232744 | intergenic | t/a | 0.48 | t | 2.72 (1.71-4.32) | NOVA1 | 95944 | 0.53 |
| rs1869995 | 3.85E−05 | 5 | 29691641 | intergenic | g/a | 0.36 | g | 2.48 (1.61-3.83) | CDH6 | 1537912 | 0.49 |
| rs891903 | 4.06E−05 | 5 | 158212216 | intronic | a/g | 0.16 | a | 3.31 (1.87-5.87) | EBF1 | 0 | 0.65 |
| rs1950420 | 5.10E−05 | 14 | 26210860 | intergenic | c/a | 0.46 | c | 2.6 (1.64-4.15) | NOVA1 | 74060 | 0.49 |
| rs7804311 | 5.64E−05 | 7 | 22225026 | intronic | t/c | 0.33 | t | 2.34 (1.55-3.53) | RAPGEF5 | 0 | 0.64 |
| rs12678719 | 5.97E−05 | 8 | 106585230 | intronic | g/c | 0.38 | g | 2.38 (1.56-3.64) | ZFPM2 | 0 | 0.009 |
| rs432793 | 6.72E−05 | 5 | 145219312 | 3′ | g/c | 0.11 | g | 0.23 (0.11-0.47) | GRXCR2 | 177 | 0.82 |
| rs10905509 | 7.41E−05 | 10 | 9160368 | intergenic | a/t | 0.29 | a | 2.47 (1.58-3.86) | GATA3 | 1003198 | 0.69 |
| rs2103738 | 7.47E−05 | 6 | 11973507 | intergenic | g/c | 0.43 | g | 2.51 (1.59-3.97) | c6orf105 | 86241 | 0.33 |
| rs17423304 | 8.11E−05 | 2 | 159978534 | intronic | g/c | 0.17 | g | 2.8 (1.68-4.68) | BAZ2B | 0 | 0.42 |
| rs10136944 | 9.29E−05 | 14 | 94930040 | intergenic | a/g | 0.2 | a | 0.33 (0.2-0.58) | C14orf139 | 13318 | 0.97 |
| rs7174597 | 9.74E−05 | 15 | 55822361 | intergenic | a/g | 0.02 | a | 0.01 (0.001-0.1) | GCOM1 | 25316 | |

### Example 2

### Analysis of AIP Candidate Genes

The present analysis focused on association of previously reported four AIP candidate genes: DAT1, DRD2, HTR2c and six candidate genes for idiopathic PD: Alpha-synuclein, Parkin, UCHL1, Pink1, DJ-1 and LRRK2. The aforementioned ten genes were selected based on literature review. As most of the reported associated variants were not genotyped in the study platform used in the present invention, and in order to study the candidate genes association systematically, all the SNP genotyped within these genes were analyzed for AIP severity. The analysis of these genes was performed in the sample obtained in Example 1.

Following correction for a number of SNPs analysed within each gene it was found that none of them associates with AIP severity (Table 5). These surprising findings highlight the advantage of the genome-wide analysis used in the present invention for identifying phenotypes associated with AIP severity.

TABLE 5
AIP association of potential AIP and PD gene candidate

| | Known | | | | | |
|---|---|---|---|---|---|---|
| | to | | | No. | P- | |
| | associate | | | of | value | Min. |
| Gene | with | Chromosome | Gene size | SNPs | <0.05 | P value |
| DAT1 | AIP | 5q15.3 | 52,629 | 10 | 1 | 0.02 |
| DRD2 | AIP | 11q23 | 65,684 | 15 | 0 | 0.07 |
| HTR2C | AIP | X | 326,073 | 11 | 0 | 0.27 |
| Alpha | PD | 4q | 112,743 | 29 | 0 | 0.18 |
| synuclein/ | | | | | | |
| PARK1 | | | | | | |
| Parkin/ | PD | 6q25 | 1,182,690 | 352 | 8 | 0.008 |
| PARK2 | | | | | | |
| UCHL1/ | PD | 4p | 11,518 | 5 | 0 | 0.11 |
| PARK5 | | | | | | |
| PINK1/ | PD | 1q36 | 18,057 | 6 | 0 | 0.1 |
| PARK6 | | | | | | |
| DJ-1/ | PD | 1p36 | 22,269 | 3 | 0 | 0.08 |
| PARK7 | | | | | | |
| LRRK2/ | PD | 12q12 | 144,274 | 29 | 2 | 0.023 |
| PARK8 | | | | | | |

### Example 3

### Genotyping RGS Genes

### Israel Study Sample

The focus of this work was on 5 RGS genes: RGS2, RGS4, RGS8, RGS9 and RGS10. The SNPs used in the study were selected based on three different databases: dbSNP, Ensembl Genome Browser and Sequenom Rea1SNP. We selected SNPs that fulfilled the following criteria: (1) located within the gene of interest or no more than 20,000 bases upstream or downstream; (2) reported heterozygosity>0.1. The heterozygosity of the selected SNPs was checked by genotyping 24 Jewish Israeli control subjects and was found appropriate. Altogether 26 SNPs fulfilled these conditions, without significant differences between Ashkenazi and non-Ashkenazi subjects. SNPs that showed significant deviation from HWE (n=2) were excluded from further analysis. A list of the 24 SNPs that were included in the analysis, with details of their location and minor allele frequency (MAF) in this sample, is provided in Table 6.

SNP genotyping was performed with a high-throughput system of chip-based mass spectrometry (matrix-assisted laser desorption/ionization time-of-flight; MALDI-TOF) (Sequenom, San Diego, Calif.). The allele determination in the sampled DNA was based on MALDI-TOF mass spectrometry of allele-specific primer products (Little et al., J Mol Medicine 1997a; 75:745-750; (Little et al., Eur J Clin Chem Clin Biochem., 1997b; 35:545-548). Genotyping assays were designed as multiplex reactions using SpectroDESIGNER software version 2.0.7 (Sequenom). Primers were synthesized by Integrated DNA Technologies (Coralville, Iowa). Optional primers that can be used for carrying out the methods of the invention are listed in Table 8 hereinbelow. The detailed PCR and primer extension reactions were according to the protocol for high multiplex homogeneous MassEXTEND (hME) procedure (Sequenom application notes, and described in McCullough et al., Nucleic Acids Res., 2005; 33: e99).

TABLE 8
Exemplary primers for detecting the SNP rs4606

| Polymorphism | | | |
|---|---|---|---|
| Nucleotide | | | SEQ |
| No. (SNP | | | ID |
| database No.) | Primer | Primer's sequence | NO: |
| 189512829 | 1-PCR | ACGTTGGATGAGTACTGATGATC | 5 |
| (rs4606) | | TGTGGTC | |
| | 2-PRC | ACGTTGGATGGGATTCAGTAACA | 6 |
| | | GTGAAGTG | |
| | UEP_SEQ | AGTGAAGTGTTTACTATGTGCAA | 7 |

The high-throughput liquid handling was performed with the aid of a MULTIMEK 96 automated 96-channel robot (Beckman Coulter, Fullerton, Calif.). Primer extension products were loaded onto a 384-element chip (SpectroCHIP; Sequenom) by nanoliter pipetting robot (SpectroPOINT, Sequenom) and analyzed with a MassARRAY mass spectrometer (Bruker Daltonik, Bremen, Germany). The resulting mass spectra were processed and analyzed for peak identification and allele determination with the MassARRAY TYPER version 3.1.4.0 software (Sequenom). About 10% of the total calls were given a low score by the Sequenom caller software, and were inspected manually for the correct call.

### U.S. Study Sample

Genomic DNA was extracted from whole blood using the Puregene® DNA purification system (Gentra Systems MA, USA). The six SNPs within or flanking the RGS2 gene (upstream and downstream) identified in the Israel sample as described herein were genotyped: rs1933695, rs2179652, rs2746073, rs4606, rs1819741 and rs1152746. (Greenbaum et al, Pharmacogenetics and Genomics, 17:519-28, 2007). Two SNPs that showed a statistically significant (P<0.05) allele frequency difference between AA and Caucasians, and were excluded from the analysis of the overall sample (rs1933695 and rs2746073). No SNPs showed significant deviation from Hardy-Weinberg equilibrium (HWE).

SNP genotyping was performed using the TaqMan Assay-On-Demand™, purchased from Applied Biosystems (Foster City, Calif., USA). The assay contains two primers and two MGB-TaqMan probes. The PCR reaction was performed according to the manufacturer's instructions. In short, 10-30 ng of gDNA were added to a reaction mixture containing 0.22 μl 20× assay reagent and 2.5 μl 2× TaqMan Universal PCR Master Mix (Applied Biosystems) in a total volume of 5 μl in 384-wells plate. PCR conditions were 2 min at 50°, 10 min at 90° and 45 cycles of 15 sec at 95° and 1 min at 60°. Real-Time PCR was performed and analyzed in an ABI PRISM 7900 HT Sequence Detection System (Applied Biosystems) with the SDS 2.3 software. For the purpose of quality control, ˜10% of the samples were genotyped twice; the match rate was 99%.

### Statistical Analysis

### Israel Study Sample

Ratings of clinical state and adverse effects were analyzed at baseline and after two weeks for patients whose treatment regimen during this time consisted of typical antipsychotics (TYP) or typical antipsychotics plus risperidone (TYP-R). SPSS version 12.01 was used to perform Student t tests, chi square tests or analysis of variance (ANOVA). The primary outcome variable for clinical response was the PANSS change score calculated by subtracting the score at two weeks from the score at baseline. For categorical analyses patients with change scores above the median were grouped as early responders (ER) and patients with scores at or below the median as non-early responders (N-ER). The primary outcomes variable for extrapyramidal symptoms of the Parkinson type was the SAS change score calculated by subtracting the score at two weeks from the score at baseline. For categorical analyses, patients demonstrating worsening of existing Parkinson symptoms during treatment or patients without parkinsonism at admission who developed it during the two weeks antipsychotic treatment were grouped as PARK+ (n=33). Patients showing improvement of existing Parkinson symptoms during treatment or patients without parkinsonism at admission and after the two weeks of antipsychotic treatment were grouped as PARK− (n=82). The same analyses were done for the BAS and AIMS. Twenty-seven patients showed onset or worsening of akathisia after two weeks of treatment with TYP or TYP-R. Only 10 patients showed onset or worsening of abnormal involuntary movements after two weeks of treatment; therefore, no further analyses were performed of results based on the AIMS.

Haploview (version 3.12) was used to examine linkage disequilibrium (LD) between SNPs, to define LD blocks and to detect significant departure from Hardy Weinberg equilibrium (HWE). Haploview was also used to perform single SNP association tests, for haplotype population frequency estimation and to perform haplotype association tests. P values<0.05 (two tailed) were regarded as nominally significant. Bonferonni correction was applied for the number of tests performed for each phenotype.

We analyzed the power of our sample to detect association of individual SNPs with early response to treatment using Power and Precision V2.0, 2000 (http://www.powerandprecision.com). The analysis was based on 240 chromosomes divided approximately equally between the ER and N-ER groups. The analysis indicated that the smallest allele frequency difference (effect size) that could be detected with 80% power (alpha 0.05, two tailed) ranged from 0.11 (95% CI: 0.03-0.19) to 0.17 (95% CI: 0.05-0.29). Given the disproportionate division of patients between the PARK+ (n=33) and PARK− groups (n=82), power was lower for this comparison. The smallest allele frequency difference that could be detected with 80% power (alpha 0.05, two tailed) ranged from 0.14 (95% CI: 0.06-0.28) to 0.20 (95% CI: 0.07-0.23). For the Akathisia+/−Akathisia− comparison the smallest allele frequency difference that could be detected with 80% power (alpha 0.05, two tailed) ranged from 0.15 (95% CI: 0.07-0.23) to 0.22 (95% CI: 0.08-0.36).

### U.S. Study Sample

For categorical analysis, patients with a SAS score of zero on two evaluation, were grouped as null (PARK−) for the phenotype of antipsychotic-induced parkinsonism (AIP) and patients whose average SAS score was above zero were grouped as positive (PARK+). To further explore association of the RGS2 gene with AIP we defined the upper quartile of patients according to SAS scores as PARK75% and compared them to PARK− patients. The same approach was used to analyze the akathisia phenotype according to the BAS. Allele and genotype frequencies were compared in PARK− vs. PARK+ and PARK75% patients and in Akathisia+ and Akathisia− patients by chi-square tests. Odds ratios and 95% confidence intervals were calculated by logistic regression, taking into account PANSS scores since these were significantly higher among PARK+ compared to PARK− patients as well as age and gender. Ethnicity and drug treatment group did not significantly influence the model and were not included.

### Example 4

### Demographic and Clinical Features at Baseline and Drug Treatment: Israel Study Sample

The first set of genetic associations that we examined was of RGS genes with response to antipsychotic treatment at two weeks. Early responders (ER) at this time point, defined by a change in scores on PANSS that exceeded the median, were compared to non-early responders (N-ER). Patients with treatment emergent or worsening Parkinsonism (PARK+) were compared with patients without treatment emergent or worsening Parkinsonism (PARK−) after two weeks of antipsychotic treatment. ER(N=61) and N-ER(N=60) did not differ in background and demographic features including age, gender, education, age at onset, age at first psychiatric hospitalization, cumulative psychiatric hospitalization, dose of typical antipsychotics and risperidone and other treatment details (Table 9). There was a trend for PANSS total scores to be slightly higher in the ER group (ER=24.5±6.4, N-ER=22.0±7.2; p=0.05). There were no significant differences between the ER and N-ER groups in allele and genotype frequency of the 24 SNPs that were tested in 5 RGS genes except for one SNP in RGS9 (rs1877822) that was nominally significant in the comparison of genotype frequency only (p=0.03).

### Example 5

### Association of RGS SNPs with EPS

Results are based on the Israel study sample. Samples from 6 of the 121 patients were not available for these analyses for technical reasons. PARK+ patients (N=33), who manifested emergent or worsening Parkinson symptoms, as defined by the SAS, were compared to patients without worsening of EPS or treatment emergent symptoms (PARK−, N=82) The two groups did not differ on background or demographic features including age, gender, education, age at onset, age at first psychiatric hospitalization, cumulative psychiatric hospitalization, dose of typical antipsychotics and risperidone and other treatment details (Table 9).

TABLE 9*
Israel Study Sample: background and demographic features

| | Feature | | | | | | | |
|---|---|---|---|---|---|---|---|---|
| | ER | | N-ER | | PARK+ | | PARK− | |
| Number | 61 | | 60 | | 33 | | 82 | |
| Age (yrs.) | 37.1 | 12.1 | 39.7 | 13.3 | 36.4 | 11.8 | 38.9 | 12.9 |
| No. of male gender (%) | 38 | — | 47 | — | 23 | — | 58 | — |
| | (65.5) | | (79.7) | | (71.9) | | (74.4) | |
| Education (yrs.) | 10.8 | 2.7 | 10.3 | 3.2 | 10.3 | 2.9 | 10.6 | 3.0 |
| Age at onset (mean, yrs) | 24.3 | 7.0 | 23.6 | 9.8 | 23.6 | 5.7 | 24.3 | 9.7 |
| Age 1st psychiatric | 24.8 | 7.2 | 24.4 | 10.7 | 24.2 | 7.9 | 24.8 | 10.0 |
| hospitalization (yrs.) | | | | | | | | |
| Cumulative psychiatric | 1.2 | 1.1 | 1.4 | 2.0 | 1.6 | 2.6 | 1.1 | 1.1 |
| hospitalization | | | | | | | | |
| (months/years at risk) | | | | | | | | |
| Typical antipsychotic | 426.0 | 412.0 | 435.2 | 234.9 | 414.46 | 481.9 | 433.4 | 339.7 |
| dose (CPZ units/day) | | | | | | | | |
| Risperidone dose | 2.9 | 1.1 | 3.3 | 2.0 | 2.75 | 0.9 | 3.2 | 1.5 |
| (mg/day) | | | | | | | | |
| 2 Typical antipsychotics | 19 | — | 17 | — | 13 | — | 23 | — |
| number (%) | (31.7) | | (28.3) | | (39.3) | | (28.2) | |
| Concomitant | 14 | — | 12 | — | 9 | — | 16 | — |
| benzodiazepines number | (23.0) | | (20.0) | | (27.3) | | (10.5) | |
| (%) | | | | | | | | |
| Concomitant | 26 | — | 23 | — | 16 | — | 31 | — |
| anticholinergics number | (42.7) | | (38.3) | | (48.5) | | (37.8) | |
| (%) | | | | | | | | |

*No comparisons, by Pearson chi square or Student t test, were significant at p < 0.1

Several SNPs in RGS genes were examined. Baseline SAS scores were similar (PARK+=12.2±3.1, PARK-=13.9±4.7). As shown in Table 7, five SNPs within or flanking the RGS2 gene were significantly associated with emergence or worsening of Parkinson symptoms over two weeks of treatment with TYP or TYP-R. Nominally significant differences in allele frequency were observed for rs2179652 (p=0.006), rs2746073 (p=0.0078), rs4606 (p=0.0008), rs1819741 (p=0.001) and rs1152746 (p=0.0455). Two of these differences (rs4606 and rs1819741) survived Bonferonni correction for the 24 tests performed (corrected alpha required, 0.001). Results for comparison of genotype frequency are also shown in Tables 6-7 and were in the same direction for RGS2. There were no nominally significant findings in the other RGS genes.

TABLE 6
SNPs examined in the RGS genes

| Gene name, | | SNP | | bp | | |
|---|---|---|---|---|---|---|
| Chromosomal | | Position | | variation | Minor | SEQ |
| location/ | Database | on | Alleles | (Major, | Allele | ID |
| Extent (bp) | SNP No. | chromosome | (The variant by is highlighted) | Minor) | Freq | NO: |
| RGS2, 1q31 | rs1933695 | 189496477 | GAATTTATGGGAGTGGATAGT | G, C | 0.14 | 8 |
| 189,509,828- | rs2179652 | 189501483 | TCCAGCCCTGTGGCCAGCCTC | T, A | 0.43 | 9 |
| 189,513,063 | rs2746073 | 189510884 | TTGGTAAAAATGCGTTCAGCT | T, A | 0.28 | 10 |
| | rs4606 | 189512829 | CTATGTGCAACGGTATTGAAG | C, G | 0.27 | 3 |
| | rs1819741 | 189516495 | GAAATAAATATACCAAATTAA | T, A | 0.29 | 11 |
| | rs1152746 | 189528562 | CTTACTGTACATGCCACAGAA | A, T | 0.18 | 12 |
| Gene name, | | SNP | | bp | | |
| Chromosomal | | Position | | variation | Minor | |
| location/ | Database | on | Alleles | (Major, | Allele | |
| Extent (bp) | SNP No. | chromosome | (the variant by is highlighted) | Minor) | Freq | |
| RGS4, 1q23.3 | rs951439 | 159765349 | | C, G | 0.43 | |
| 159,770,809- | rs6678136 | 159768975 | | G, C | 0.43 | |
| 159,778,040 | rs2842030 | 159772153 | | T, A | 0.48 | |
| | rs10759 | 159778009 | | C, G | 0.24 | |
| | rs2063142 | 159784947 | | T, A | 0.17 | |
| RGS8, 1q25 | rs3845459 | 179352555 | | T, A | 0.37 | |
| 179,347,449- | rs2023596 | 179354668 | | A, T | 0.37 | |
| 179,373,706 | rs4651129 | 179357234 | | A, T | 0.37 | |
| | rs4652741 | 179358395 | | A, T | 0.38 | |
| | rs567397 | 179372295 | | A, T | 0.08 | |
| RGS9, | rs1877822 | 60595539 | | T, A | 0.35 | |
| 17q23-q24 | rs2869578 | 60610219 | | C, G | 0.28 | |
| 60,564,054- | | | | | | |
| 60,654,270 | | | | | | |
| RGS10, | rs3009892 | 121239161 | | G, C | 0.29 | |
| 10q25 | rs756279 | 121249869 | | A,T | 0.10 | |
| 121,249,442- | rs7919216 | 121255625 | | T, A | 0.09 | |
| 121,292,157 | rs1556591 | 121265893 | | A, T | 0.29 | |
| | rs1467813 | 121271597 | | G, C | 0.34 | |
| | rs7071853 | 121301596 | | T, A | 0.26 | |

TABLE 7
SNPs associated with EPS

| | | | PARK+ |
|---|---|---|---|
| | | PARK+ | vs. |
| Gene name, | | vs. | PARK− |
| Chromosomal location/ | | PARK− | p Genotype |
| Extent (bp) | dbSNP No. | p Allele (df 1) | (df 2) |
| RGS2, 1q31 | rs1933695 | 0.439 | 0.800 |
| 189, 509, 828-189, | rs2179652 | 0.006 | 0.032 |
| 513, 063 | rs2746073 | 0.008 | 0.061 |
| | rs4606 | 0.0008* | 0.007* |
| | rs1819741 | 0.001* | 0.029 |
| | rs1152746 | 0.045 | 0.040 |
| RGS4, 1q23.3 | rs951439 | 0.901 | 0.756 |
| 159, 770, 809-159, | rs6678136 | 0.816 | 0.961 |
| 778, 040 | rs2842030 | 0.904 | 0.592 |
| | rs10759 | 0.561 | 0.802 |
| | rs2063142 | 0.787 | 0.864 |
| RGS8, 1q25 | rs3845459 | 0.749 | 0.949 |
| 179, 347, 449-179, | rs2023596 | 0.521 | 0.962 |
| 373, 706 | rs4651129 | 0.502 | 0.568 |
| | rs4652741 | 0.490 | 0.512 |
| | rs567397 | 0.057 | 0.046 |
| RGS9, 17q23-q24 | rs1877822 | 0.730 | 0.604 |
| 60, 564, 054-60, 654, 270 | rs2869578 | 0.567 | 0.880 |
| RGS10, 10q25 | rs3009892 | 0.736 | 0.854 |
| 121, 249, 442-121, | rs756279 | 0.670 | 0.723 |
| 292, 157 | rs7919216 | 0.895 | 0.917 |
| | rs1556591 | 0.440 | 0.576 |
| | rs1467813 | 0.624 | 0.890 |
| | rs7071853 | 0.423 | 0.604 |

*Survives Bonferonni correction

### Example 6

### Association of the RGS2 SNPs with Development of EPS in the U.S. Study Sample

There were no significant differences between PARK+ (n=141) and PARK− patients (n=43) as regards demographic and clinical data, such as age, sex, ethnic origin (AA or Caucasian) and type of antipsychotic treatment (Table 10). However, mean PANSS score was significantly higher among PARK+ compared to PARK− patients (P=0.00002) and PARK75% compared to PARK− patients (P=0.001). A similar difference was observed in the African-American sub-sample (PARK+ vs. PARK−, P=0.004; PARK75% vs. PARK−, P=0.015).

TABLE 10
Demographic and clinical features of patients with (PARK+) or without (PARK−) antipsychotic
induced Parkinsonism, assessed by the Simpson Angus Scale (SAS)

| | A. Whole Sample | | | | B. African American sub-sample | | | |
|---|---|---|---|---|---|---|---|---|
| | PARK+ | | PARK− | | PARK+ | | PARK− | |
| | Mean/n | | Mean/n | | Mean/n | | Mean/n | |
| | (%) | SD | (%) | SD | (%) | SD | (%) | SD |
| Number | 141 (76.6) | | 43 (23.4) | | 83 (74.1) | | 29 (25.9) | |
| Age | 40.3 | 9.8 | 39.9 | 9.1 | 40.29 | 9.7 | 41.1 | 8.8 |
| Male Gender | 129 (93.5) | | 40 (95.2) | | 79 (95.2) | | 27 (93.1) | |
| Antipsychotic | | | | | | | | |
| Treatment¹ | | | | | | | | |
| Typical | 36 (25.5) | | 8 (18.6) | | 23 (27.7) | | 7 (24.1) | |
| Risperidone | 32 (22.7) | | 14 (32.6) | | 23 (27.8) | | 10 (34.5) | |
| Olanzapine | 37 (26.2) | | 12 (27.9) | | 20 (24.1) | | 7 (24.1) | |
| Clozapine | 33 (23.4) | | 8 (18.6) | | 17 (20.5) | | 5 (17.2) | |
| Rating Scales | | | | | | | | |
| PANSS total | 70.7** | 17.3 | 59.9 | 12.0 | 68.7* | 15.2 | 59.4 | 12.4 |
| SAS total | 2.70** | 2.83 | 0 | 0 | 2.59** | 1.81 | 0 | 0 |
| BAS total | 1.028* | 1.649 | 0.314 | 1.05 | 0.687 | 1.175 | 0.397 | 1.256 |
| Ethnicity | | | | | | | | |
| African American | 83 (58.9) | | 29 (67.4) | | | | | |
| Hispanic | 30 (21.3) | | 11 (25.6) | | | | | |
| White | 25 (17.7) | | 2 (4.7) | | | | | |
| Caucasian² | 55 (39.9) | | 13 (31) | | | | | |

*p < 0.01
**p < 0.0001
¹Drug information missing for 3 patients in PARK+ and 1 in PARK− in the overall sample
²Hispanic + White
PANSS—Positive and Negative Symptoms scale;
SAS—Simpson Angus scale;
BAS—Barnes Akathisia scale

Of the 4 SNPs that were suitable for association testing in the sample as a whole (no allele frequency differences between African American and Caucasian patients), one (rs4606), was associated with AIP (P=0.033) at a nominally significant level, the minor (G) allele being more frequent in the PARK− compared to the PARK+ group (Table 11A). When comparing the PARK− and the PARK75% groups, the significance level was stronger for rs4606 (P=0.016) and rs181974 emerged as nominally significant (P=0.046) (Table 11 B).

TABLE 11A
SNP association with Parkinsonism

| Alleles | | | | |
|---|---|---|---|---|
| Major, | MinAF | MinAF | PARK+ vs. PARK− | PARK+ vs. PARK− |
| Minor | PARK+ | PARK− | P Allele (d.f. 1) | P Genotype (d.f. 2) |
| T, C | 0.44 | 0.42 | 0.697 | 0.633 |
| C, G | 0.27 | 0.39 | 0.033 | 0.043 |
| T, C | 0.24 | 0.33 | 0.091 | 0.169 |
| A, G | 0.25 | 0.34 | 0.116 | 0.241 |
| G, A | 0.04 | 0.07 | 0.372 | 0.286 |
| T, C | 0.42 | 0.37 | 0.505 | 0.410 |
| T, A | 0.08 | 0.07 | 0.919 | 0.809 |
| C, G | 0.27 | 0.43 | 0.027 | 0.023 |
| T, C | 0.22 | 0.36 | 0.046 | 0.040 |
| A, G | 0.27 | 0.30 | 0.654 | 0.575 |

TABLE 11B
SNP Association with Parkinsonism

| Alleles | | PARK− 75% vs. | |
|---|---|---|---|
| Major, | MinAF | PARK− | PARK− 75% vs.PARK− |
| Minor | PARK 75% | P Allele (d.f. 1) | P genotype (d.f 2) |
| T, C | 0.45 | 0.643 | 0.847 |
| C, G | 0.24 | 0.016 | 0.021 |
| T, C | 0.20 | 0.026 | 0.064 |
| A, G | 0.31 | 0.613 | 0.817 |
| G, A | | | |
| T, C | 0.05 | 0.628 | 0.460 |
| T, A | 0.37 | 0.967 | 1.000 |
| C, G | 0.05 | 0.592 | 0.439 |
| T, C | 0.23 | 0.025 | 0.027 |
| A, G | 0.18 | 0.034 | 0.024 |

### Example 7

### The Protective Effect of rs4606 Against AIP

Logistic regression analysis, controlling for age, gender and PANSS scores (Table 12) emphasizes the significant protective effect of the rs4606 G allele against AIP in the Israeli and US samples and the African-American sub-sample. Carriers of the rs4606 G allele (as heterozygotes or homozygotes) were 3.2-5.3 times less likely to be in the PARK+ group. The effect of the TGCA haplotype was similar but weaker, particularly in the Israeli sample.

In contrast to AIP, no association of SNPs or haplotypes in the RGS2 gene with antipsychotic-induced akathisia, as measured by the BAS, was observed.

TABLE 12
Protective effect of RGS2-rs4606 G allele against antipsychotic-
induced Parkinsonism

| | | | 1/Odds | | p |
|---|---|---|---|---|---|
| | PARK+ | PARK− | Ratio | 95% CI | Value |
| rs4606 G Allele Carriers | | | | | |
| Israeli Sample | 0.25 | 0.51 | 3.03 | −0.12 | 0.034 |
| | | | | 0.92 | |
| US Overall Sample* | 0.45 | 0.67 | 4.50 | −0.01 | 0.001 |
| | | | | 0.52 | |
| African-American Sub- | 0.43 | 0.71 | 5.26 | −0.07 | 0.002 |
| Sample* | | | | 0.56 | |
| TGCA Haplotype Carriers | | | | | |
| Israeli Sample | 0.27 | 0.49 | 2.44 | −0.16 | 0.079 |
| | | | | 1.10 | |
| US Overall Sample* | 0.38 | 0.58 | 3.23 | −0.14 | 0.003 |
| | | | | 0.66 | |
| African-American Sub- | 0.32 | 0.59 | 4.17 | −0.09 | 0.004 |
| Sample* | | | | 0.63 | |

*Analysis control for PNASS scores

EXAMPLE 8

### A Combination of Two Specific SNPs is Predictive of Resistance to AIP

The utility of specific genetic markers within the RGS2 and ZFPM2 genes as biomarkers for susceptibility to antipsychotic induced parkinsonism (AIP) was determined in a sample of Jewish schizophrenia patients treated with antipsychotic medication for at least one month.

Prior results showed in two different samples that schizophrenia patients who carry the G allele of SNP rs4606 in the 3′ untranslated region of the RGS2 gene are significantly less likely to manifest AIP when treated with antipsychotic drugs than non-carriers of this allele (Greenbaum et al, 2007, ibid; Greenbaum et al, Pharmacogenomics J., 9(2):103-110, 2008).

It was further shown that SNP rs12678719 in the ZFPM2 gene is associated with susceptibility to AIP. It was found that carriers of the C allele of the intronic rs12678719 SNP were less likely to manifest AIP than non-carriers (Alkelai et al, ibid) and replicated this finding in a second sample (Greenbaum et al, Psychopharmacology (Berlin), to be submitted).

Considering the two genetic variants together, i.e. RGS2-G allele carrier or ZFPM2-C allele carrier or carrier of both, yielded a positive predictive value (PPV)₁ of 0.71 and a negative predictive value (NPV)² of 0.68 with odds ratio (OR)₃ of 5.13 for prediction of susceptibility to antipsychotic induced parkinsonism.

These findings establish that methods and diagnostic kits including means for detecting SNP rs4606 in RGS2 gene and means for detecting the SNP rs12678719 ZFPM2 gene have clinical utility in identifying patients who are at lesser risk for developing AIP when prescribed antipsychotic drugs.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A method for assessing resistance of a subject to develop antipsychotic-induced parkinsonism, comprising:
(a) obtaining a sample comprising genetic material from the subject;
(b) determining in said genetic material the presence of the nucleotide sequence of the ZFPM2 gene or a fragment thereof; and
(c) identifying in said nucleotide sequence the polymorphic site rs12678719,
wherein the presence of cytosine at rs12678719 is indicative of resistance to emergence or aggravation of antipsychotic-induced parkinsonism.

2. The method of claim 2, further comprising determining in said genetic material the presence of the nucleotide sequence of the RGS2 gene or a fragment thereof; and identifying in said nucleotide sequence the polymorphic site rs4606, wherein the presence of guanine at rs4606 is indicative of resistance to emergence or aggravation of antipsychotic-induced parkinsonism.

3. The method of claim 1, comprising determining in said genetic material the presence of a first nucleotide sequence comprising the ZFPM2 gene or a fragment thereof and a second nucleotide sequence comprising the RGS2 gene or a fragment thereof; and identifying in said first and second nucleotide sequences the polymorphic sites rs12678719 and rs4606, respectively, wherein the presence of cytosine at rs12678719 or guanine at rs4606 is indicative of resistance to emergence or aggravation of antipsychotic-induced parkinsonism.

4. The method of claim 3, wherein the identity of cytosine at rs12678719 and guanine at rs4606 is indicative of resistance to emergence or aggravation of antipsychotic-induced parkinsonism.

5. The method of claim 1, wherein determining the presences of the polymorphic site comprises amplifying the genetic locus encompassing said polymorphic site.

6. The method of claim 1, wherein the sample is obtained from a biological specimen selected from the group consisting of: blood, saliva, urine, sweat, buccal material, skin and hair.

7. The method of claim 1, wherein the subject in need thereof is psychotic.

8. The method of claim 9, wherein the subject in need thereof is diagnosed with schizophrenia.

9. The method of claim 1, wherein diagnosing the resistance to emergence or aggravation of antipsychotic-induced parkinsonism is performed prior to or following treatment with one or more antipsychotic drug.

10. (canceled)

11. The method of claim 9, wherein the one or more antipsychotic drug is selected from the group consisting of: perphenazine, olanzapine, clozapine, quetiapine, risperidone and ziprasidone.

12. The method of claim 1, further comprising repeating steps (b) and (c).

13. The method of claim 1, further comprising amplifying said nucleotide sequence of the gene or fragment thereof prior to step (c).

14. The method of claim 1, wherein the antipsychotic-induced parkinsonism comprises one or more of bradykinesia, tremor, rigidity, stooped posture, gait disturbance, salivation and seborrheic dermatitis.

15. The method of claim 3, wherein assessing resistance of a subject to develop antipsychotic-induced parkinsonism is assessing resistance of a subject to develop extrapyramidal symptoms upon treatment with one or more antipsychotic drugs, wherein the presence of at least one of cytosine at rs12678719 and guanine at rs4606 is indicative of resistance to emergence or aggravation of extrapyramidal symptoms induced by treatment with the one or more antipsychotic drugs.

16.-25. (canceled)

26. A kit for assessing resistance of a subject to develop API, comprising oligonucleotides for amplification of the genetic locus encompassing the polymorphic site rs12678719 in the gene ZFPM2 within a sample obtained from the subject.

27. The kit of claim 26, further comprising means for determining the presence of the ‘c’ allele of rs12678719.

28. The kit of claim 26, further comprising oligonucleotides for amplification of the genetic locus encompassing the polymorphic site rs4606 in the gene RGS2 within said sample.

29. The kit of claim 28, further comprising means for determining the presence of the ‘g’ allele of rs4606.

30. The kit of claim 26, wherein the sample is obtained from a biological specimen selected from the group consisting of: blood, saliva, urine, sweat, buccal material, skin and hair.

31.-32. (canceled)


SEQUENCE LISTING (12 sequences)

SEQ ID NO: 1 - DNA, 601 bp, Homo sapiens
ataggaaaaaagactggcctggctctatgcattgggaacagactccaaaatatcagtgagccacacttgatatctgtgagcagggttatgtgatattattaatgaactaaccagagtgtgccatgaaagcttaacatactttaaagtatttgtagagagaaaataataaacactattagcacatagttccaagcacccttaaggaaggtactatttactcacattgataaagtcttatttaagctatttgactagaaaaggtatgtggtagaggaggaaaagtgtttttattataactgaccctaacaatgacctagaaaacaatttgaaatgattgaatgatgaacttgattccttggcttgcagtagaagaattttaaggtttattaatcactgcagtcgctgctggtatccattctgttttgtggcatcatttgaaccccatgatttcatgaataacattcccctgcagatagttggttggagctgcccttattttttaaatgtttgtattctgtggcttgtcaagaagtaaggaacagggctttcttagagataactcccctaggattaaatccaagggagtggttcttaaacattt

SEQ ID NO: 2 - DNA, 601 bp, Homo sapiens
ataggaaaaaagactggcctggctctatgcattgggaacagactccaaaatatcagtgagccacacttgatatctgtgagcagggttatgtgatattattaatgaactaaccagagtgtgccatgaaagcttaacatactttaaagtatttgtagagagaaaataataaacactattagcacatagttccaagcacccttaaggaaggtactatttactcacattgataaagtcttatttaagctatttgactagaaaaggtatgtggtagaggaggaaaagtgtttttattataactgagcctaacaatgacctagaaaacaatttgaaatgattgaatgatgaacttgattccttggcttgcagtagaagaattttaaggtttattaatcactgcagtcgctgctggtatccattctgttttgtggcatcatttgaaccccatgatttcatgaataacattcccctgcagatagttggttggagctgcccttattttttaaatgtttgtattctgtggcttgtcaagaagtaaggaacagggctttcttagagataactcccctaggattaaatccaagggagtggttcttaaacattt

SEQ ID NO: 3 - DNA, 21 bp, Homo sapiens
ctatgtgcaacggtattgaag

SEQ ID NO: 4 - DNA, 21 bp, Homo sapiens
ctatgtgcaagggtattgaag

SEQ ID NO: 5 - DNA, 30 bp, Artificial Sequence
  PCR primer
acgttggatgagtactgatgatctgtggtc

SEQ ID NO: 6 - DNA, 31 bp, Artificial Sequence
  pcr primer
acgttggatgggattcagtaacagtgaagtg

SEQ ID NO: 7 - DNA, 23 bp, artificial
  pcr primer
agtgaagtgtttactatgtgcaa

SEQ ID NO: 8 - DNA, 21 bp, Homo sapiens
gaatttatgggagtggatagt

SEQ ID NO: 9 - DNA, 21 bp, Homo sapiens
tccagccctgtggccagcctc

SEQ ID NO: 10 - DNA, 21 bp, Homo sapiens
ttggtaaaaatgcgttcagct

SEQ ID NO: 11 - DNA, 21 bp, Homo sapiens
gaaataaatataccaaattaa

SEQ ID NO: 12 - DNA, 21 bp, Homo sapiens
cttactgtacatgccacagaa